(19) United States
(12) Patent Application Publication
(10) Pub. No.: US 2013/0296279 A1
(43) Pub. Date: Nov. 7, 2013

(54) SUBSTITUTED TETRACYCLINES

(71) Applicant: Concert Pharmaceutcals Inc., Lexington, MA (US)

(72) Inventor: Adam Morgan, Ashland, MA (US)

(21) Appl. No.: 13/892,031

(22) Filed: May 10, 2013

Related U.S. Application Data
(63) Continuation of application No. PCT/US2011/060310, filed on Nov. 11, 2010.
(60) Provisional application No. 61/412,432, filed on Nov. 11, 2010.

(51) Int. Cl. C07C 237/26; C07D 205/04; C07D 207/16; C07D 207/09; C07D 221/18

(52) U.S. Cl. CPC C07C 237/26; C07D 207/09; C07D 221/18; C07D 207/16; C07D 205/04
USPC 514/152; 552/203; 548/567; 514/428; 546/78; 514/284; 552/205; 548/537; 514/423; 548/953

(57) ABSTRACT

The invention in one embodiment is directed to a compound of formula (I)

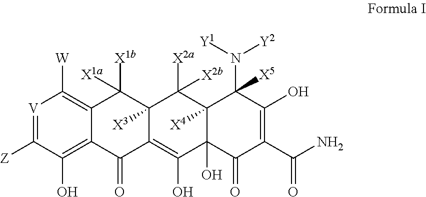

or a pharmaceutically acceptable salt thereof. The invention is also directed to a composition comprising the compound of formula I or a pharmaceutically acceptable salt, and methods of treating the indications listed herein.

### CROSS-REFERENCE TO RELATED APPLICATIONS

This application claims priority to U.S. Provisional Application Ser. No. 61/412,432, filed on Nov. 11, 2010, the content of which is incorporated by reference in its entirety.

### BACKGROUND OF THE INVENTION

Many current medicines suffer from poor absorption, distribution, metabolism and/or excretion (ADME) properties that prevent their wider use. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to maintain a sufficiently high plasma level of drug. This, however, introduces a number of potential treatment problems such as poor patient compliance with the dosing regimen, side effects that become more acute with higher doses, and increased cost of treatment.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, quinidine has been added to dextromethorphan for the purpose of reducing rapid CYP2D6 metabolism in a treatment of pseudobulbar affect. Quinidine, however, is a CYP2D6 inhibitor that has unwanted side effects that greatly limit its use in potential combination therapy (see Wang, L et al., Clinical Pharmacology and Therapeutics, 1994, 56(6 Pt 1): 659-67; and FDA label for quinidine at wvvw.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism. (See Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of overall metabolism will differ from that of its undeuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

This invention relates to novel deuterated and fluorinated tetracycline analogs. Tetracyclines serve as bacteriostatic agents which inhibit bacterial protein synthesis by binding the 30S ribosomal subunit blocking access to aminoacyl-tRNA. Compounds of Formula I are useful for the treatment of a wide range of both gram-positive and gram-negative bacterial infections including methicillin-resistant Staphylococcus aureus (MRSA). Examples of tetracyclines include tigecycline ((4S,4a S,5a R,12a S)-9-[2-(tert-butylamino)acetamido]-4,7-bis(dimethylamino)-1,4,4a,5,5a,6,11,12a-octahydro-3,10,12,12a-tetrahydroxy-1,11-dioxo-2-naphthacenecarboxamide), which is approved as Tygacil® for the treatment of complicated skin and skin structure infections (cSSSI), complicated intra-abdominal infections and community-acquired bacterial pneumonia, and omadacycline ((4S,4aS,5aR,12aS)-4,7-bis(dimethylamino)-9-(2,2-dimethylpropylaminomethyl)-3,10,12,12a-tetrahydroxy-1,11-dioxo-1,4,4a,5,5a,6,11,12a-octahydrotetracene-2-carboxamide) which is currently in clinical trials. Tigecycline is currently undergoing clinical evaluation for treatment of biliary liver cirrhosis; hospital-acquired pneumonia; rapidly growing mycobacterial lung disease; diabetic foot infections; intra-abdominal infection including appendicitis, cholecystitis, diverticulitis, intra-abdominal abscess, and peritonitis; and tunneled hemodialysis catheter-associated bacteremia (see http://clinicaltrials.gov). Omadacycline is currently undergoing clinical evaluation for treatment of complicated skin and skin structure infections (cSSSI) (see http://clinicaltrials.gov).

Despite the beneficial activities of tetracyclines, there is a continuing need for new compounds that have beneficial effects for treatment of bacterial infections.

### DEFINITIONS

The term “treat” means decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

The term “alkyl” refers to a monovalent saturated hydrocarbon group. For example, C₁-C₆ alkyl is an alkyl having from 1 to 6 carbon atoms. An alkyl may be linear or branched. Examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl and 2-methylpentyl.

The term “alkylene” refers to a divalent saturated hydrocarbon group. C₁-C₆ alkylene is an alkylene having from 1 to 6 carbon atoms. An alkylene may be linear or branched. Examples of alkylene groups include —CH₂—; —CH₂—CH₂—; —CH(CH₃)—; —CH₂—CH₂—CH₂—; —CH(CH₃)—CH₂—; —C(CH₃)₂—; —CH₂—CH₂—CH₂—CH₂—; and the like.

The term “cycloalkyl” refers to a monocyclic or bicyclic—which may be fused, bridged, or spiro-monovalent saturated or unsaturated or non-aromatic hydrocarbon ring system of 3-10 carbon atoms, such as 3 to 8 carbon atoms; the latter is denoted as C₃-C₈ cycloalkyl. Examples of cycloalkyl groups include cyclopropyl, cyclobutyl, cyclopentyl, cyclohexyl, cycloheptyl, norbornyl, spiro[4.5]decanyl, and the like.

“Heterocyclyl” refers to a monocyclic or bicyclic—which may be fused, bridged, or spiro-3- to 10-membered monovalent saturated or unsaturated non-aromatic ring system containing from 1 to 4 ring heteroatoms independently selected from N, O, and S. Exemplary heterocyclyl groups include azepanyl, azetidinyl, aziridinyl, imidazolidinyl, morpholinyl, oxazolidinyl, oxazolidinyl, piperazinyl, piperidinyl, pyrazolidinyl, pyrrolidinyl, quinuclidinyl, tetrahydrofuranyl, and thiomorpholinyl.

The term “halo” or “halogen” refers to —Cl, —Br, —F, and —I.

It will be recognized that some variation of natural isotopic abundance occurs in a synthesized compound depending upon the origin of chemical materials used in the synthesis. Thus, a preparation of a tetracycline, such as tigecycline or asomadacycline, will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention. A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any salt that is non-toxic upon administration to a recipient at a therapeutically effective dose level, and is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free of another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes specified herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert” and “t-” each refer to tertiary. “US” refers to the United States of America.

The phrase “substituted with deuterium” means that one or more positions in the indicated moiety are substituted with a deuterium atom.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

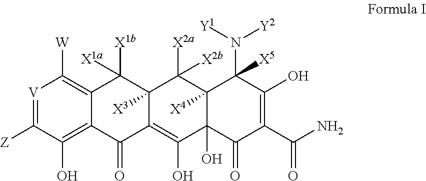

or a pharmaceutically acceptable salt thereof, wherein:

### V is CH or N;

### W is Cl, F, N(CH₃)₂, N(CD₃)₂, CH₃, CD₃, OCH₃, or OCD₃;

each X is independently selected from H and D;
each Y is independently selected from CH₃ and CD₃;
Z is H; NH₂; C₁-C₆ alkylene-R¹ wherein the C₁-C₆ alkylene is optionally substituted with deuterium; or NHQR¹;
Q is —C(O)— or a direct bond;
R¹ is C₀-C₆ alkyl optionally substituted with deuterium and optionally substituted with R⁴; or NH(C₁-C₆ alkyl) wherein the C₁-C₆ alkyl is optionally substituted with deuterium; R⁴ is NR²R³; or a 3- to 10-membered heterocyclyl containing at least one ring nitrogen wherein the 3-10-membered heterocyclyl is optionally substituted with deuterium at a carbon atom and is optionally substituted with C₁-C₆ alkyl that is optionally substituted with deuterium;
each of R² and R³ is independently H; C₁-C₆ alkyl; (C₃-C₈ cycloalkyl); or (C₀-C₂)alkylene(C₃-C₈ cycloalkyl); wherein each C₁-C₆ alkyl, (C₃-C₈ cycloalkyl), and (C₀-C₂)alkylene(C₃-C₈ cycloalkyl) of R² and R³ is independently optionally substituted with deuterium;
with the proviso that if W is other than CD₃, OCD₃ or N(CD₃)₂; each X is H; and each Y is CH₃; then Z comprises deuterium;
and with the proviso that if W is N(CH₃)₂ or N(CD₃)₂, then X⁵ is D and at least one of X^{1a}, X^{1b}, X^{2a}, X^{2b}, X³ and X⁴ is hydrogen.

In one embodiment, X^{1a} and X^{1b} are the same; X^{2a} and X^{2b} are the same; Y¹ and Y² are the same; and Z is H; NH₂; C₁-C₅ alkylene-R¹ where the C₁-C₅ alkylene is optionally substituted with deuterium; or NHQR¹.

In one embodiment, X⁵ is deuterium. In one aspect of this embodiment, X^{1a} and X^{1b} are each hydrogen. In another aspect, X^{1a} and X^{1b} are each deuterium.

In one embodiment, V is CH. In one aspect of this embodiment, W is F, N(CH₃)₂, or N(CD₃)₂; X^{1a} and X^{1b} are the same; X^{2a} and X^{2b} are the same; Y¹ and Y² are the same. In an example of this aspect, X^{1a}, X^{1b}, X^{2a} and X^{2b} are each hydrogen. In a more particular example of this aspect, Z is H, (C₁-C₆)alkyleneNH(C₁-C₆ alkyl), NHCOC₁-C₆ alkyl, NHCO(C₁-C₆)alkyleneNH(C₁-C₆ alkyl), NHCO(C₁-C₆)alkyleneNH(C₀-C₂)alkylene-(C₃-C₈ cycloalkyl), NHCO(C₁-C₆)alkyleneN(C₁-C₆ alkyl)(C₁-C₆ alkyl), NHC(O) (C₀-C₂)alkylene-(3- to 10-membered heterocyclyl optionally substituted with C₁-C₄ alkyl), wherein Z is optionally substituted with deuterium. In an even more particular example, Z is H, CH₂NHCH₂C(CH₃)₃, NHCOCH₂C(CH₃)₃, NHCOCH₂NHCH₂CH₃, NHCOCH₂NHCH₂CH₂CH₃, NHCOCH₂NHCH₂CH₂CH₂CH₃, NHCOCH₂NH-cyclopentyl, NHCOCH₂NH-cyclobutyl, NHCOCH₂NHCH₂-cyclopropyl, NHCOCH₂NHCH₂-cyclobutyl, NHC(O)CH₂N(CH₃)₂, NHC(O)CH₂N(CH₂CH₃)(CH₃), NHC(O)CH₂(1-pyrrolidyl), NHCO—(S)-2-pyrrolidyl, NHCO—(S)-2-azetidinyl, NHCO—(S)-2-(N-methyl)-azetidyl, or NHCO—(S)-2-(N-methyl)-pyrrolidyl, wherein Z is optionally substituted with deuterium.

In another embodiment, V is N. In one aspect of this embodiment, W is Cl, F, N(CH₃)₂, N(CD₃)₂, OCH₃ or OCD₃; X^{1a} and X^{1b} are the same; X^{2a} and X^{2b} are the same; Y¹ and Y² are the same. In an example of this aspect, X^{1a}, X^{1b}, X^{2a} and X^{2b} are each hydrogen.

In a more particular example of this aspect, Z is NH₂, NH(C₁-C₆ alkyl), NH(C₁-C₆)alkyleneNH(C₁-C₆ alkyl), NH(C₁-C₆)alkyleneN(C₁-C₆ alkyl)(C₁-C₆ alkyl), NH(C₁-C₆)alkylene-(3- to 10-membered heterocyclyl optionally substituted with C₁-C₄ alkyl), wherein Z is optionally substituted with deuterium. In an even more particular example of this aspect, Z is NH₂, NHCH₂CH₃, NHCH₂CH₂CH₃, NHCH₂C(CH₃)₂CH₂N(CH₃)₂, NHCH₂C(CH₃)₂CH₂(1-pyrrolidyl), wherein Z is optionally substituted with deuterium.

In one embodiment, the compound of formula I is a compound of formula Ia

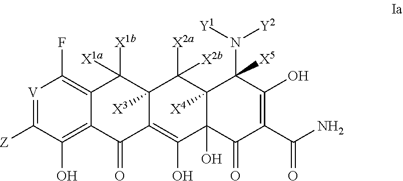

or pharmaceutically acceptable salt thereof. In one aspect of this embodiment, V is CH; X^{1a} and X^{1b} are the same; X^{2a} and X^{2b} are the same; Y¹ and Y² are the same. In an example of this aspect, X^{1a}, X^{1b}, X^{2a} and a X^{2b} are each hydrogen. In a more particular example of this aspect, Z is H, (C₁-C₆)alkyleneNH(C₁-C₆ alkyl), NHCOC₁-C₆ alkyl, NHCO(C₁-C₆)alkyleneNH(C₁-C₆ alkyl), NHCO(C₁-C₆)alkyleneNH(C₀-C₂)alkylene-(C₃-C₈ cycloalkyl), NHCO(C₁-C₆)alkyleneN(C₁-C₆ alkyl)(C₁-C₆ alkyl), or NHC(O) (C₀-C₂)alkylene-(3- to 10-membered heterocyclyl optionally substituted with C₁-C₄ alkyl), wherein Z is optionally substituted with deuterium.
In an even more particular example of this aspect, Z is H, CH₂NHCH₂C(CH₃)₃, NHCOCH₂C(CH₃)₃, NHCOCH₂NHCH₂CH₃, NHCOCH₂NHCH₂CH₂CH₃, NHCOCH₂NHCH₂CH₂CH₂CH₃, NHCOCH₂NH-cyclopentyl, NHCOCH₂NH-cyclobutyl, NHCOCH₂NHCH₂-cyclopropyl, NHCOCH₂NHCH₂-cyclobutyl, NHC(O)CH₂N(CH₃)₂, NHC(O)CH₂N(CH₂CH₃)(CH₃), NHC(O)CH₂(1-pyrrolidyl), NHCO—(S)-2-pyrrolidyl, NHCO—(S)-2-azetidinyl, NHCO—(S)-2-(N-methyl)-azetidyl, or NHCO—(S)-2-(N-methyl)-pyrrolidyl, wherein Z is optionally substituted with deuterium. In another aspect of this embodiment, V is N; X^{1a} and X^{1b} are the same; X^{2a} and X^{2b} are the same; Y¹ and Y² are the same. In an example of this aspect, X^{1a}, X^{1b}, X^{2a} and X^{2b} are each hydrogen. In a more particular example of this aspect, Z is NH₂, NH(C₁-C₆ alkyl), NH(C₁-C₆)alkyleneNH(C₁-C₆ alkyl), NH(C₁-C₆)alkyleneN(C₁-C₆ alkyl)(C₁-C₆ alkyl), or NH(C₁-C₆)alkylene-(3- to 10-membered heterocyclyl optionally substituted with C₁-C₄ alkyl), wherein Z is optionally substituted with deuterium. In an even more particular example of this aspect, Z is NH₂, NHCH₂CH₃, NHCH₂CH₂CH₃, NHCH₂C(CH₃)₂CH₂N(CH₃)₂, NHCH₂C(CH₃)₂CH₂(1-pyrrolidyl), wherein Z is optionally substituted with deuterium.

In one aspect of any of the preceding embodiments, the R⁴ 3-10 membered heterocyclyl is (1 pyrrolidyl), (2-(S)-pyrrolidyl), (2-(S)-azetidyl), or (2-(S)—N-methyl-azetidyl), each optionally substituted with deuterium at a carbon atom.

In another set of embodiments, any atom not designated as deuterium in any of the embodiments set forth above is present at its natural isotopic abundance.

Specific examples of compounds of Formula I include the compounds in the tables below. In all tables, unless otherwise specified, any atom not designated as deuterium in substituent Z is present at its natural isotopic abundance.

Specific examples of a compound of Formula I include one of the compounds of Table 1 or a pharmaceutically acceptable salt thereof, wherein V is CH; each of X^{2a}, X^{2b}, X³ and X⁴ is hydrogen, and Z is hydrogen:

TABLE 1

| Cmpd | W | X^{1a} | X^{1b} | X⁵ | Y¹ | Y² |
|---|---|---|---|---|---|---|
| 100 | F | H | H | D | CH₃ | CH₃ |
| 101 | F | H | H | D | CD₃ | CD₃ |
| 102 | F | H | H | H | CD₃ | CD₃ |
| 103 | F | D | D | D | CD₃ | CD₃ |
| 104 | F | D | D | D | CH₃ | CH₃ |
| 105 | F | D | D | H | CH₃ | CH₃ |
| 106 | F | D | D | H | CD₃ | CD₃ |
| 107 | N(CH₃)₂ | H | H | D | CH₃ | CH₃ |
| 108 | N(CH₃)₂ | H | H | D | CD₃ | CD₃ |
| 109 | N(CH₃)₂ | H | H | H | CD₃ | CD₃ |
| 110 | N(CH₃)₂ | D | D | D | CD₃ | CD₃ |
| 111 | N(CH₃)₂ | D | D | D | CH₃ | CH₃ |
| 112 | N(CH₃)₂ | D | D | H | CH₃ | CH₃ |
| 113 | N(CH₃)₂ | D | D | H | CD₃ | CD₃ |
| 114 | N(CD₃)₂ | H | H | D | CH₃ | CH₃ |
| 115 | N(CD₃)₂ | H | H | D | CD₃ | CD₃ |
| 116 | N(CD₃)₂ | H | H | H | CD₃ | CD₃ |
| 117 | N(CD₃)₂ | D | D | D | CD₃ | CD₃ |
| 118 | N(CD₃)₂ | D | D | D | CH₃ | CH₃ |
| 119 | N(CD₃)₂ | D | D | H | CH₃ | CH₃ |
| 120 | N(CD₃)₂ | D | D | H | CD₃ | CD₃ |
| 121 | N(CD₃)₂ | H | H | H | CH₃ | CH₃ |

Specific examples of a compound of Formula I include one of the compounds of Table 2 or a pharmaceutically acceptable salt thereof, wherein V is CH; each of X^{2a}, X^{2b}, X³ and X⁴ is hydrogen, and Z is NHCOCH₂NHCH₂CH₂CH₃:

TABLE 2

| Cmpd | W | X^{1a} | X^{1b} | X⁵ | Y¹ | Y² |
|---|---|---|---|---|---|---|
| 122 | F | H | H | D | CH₃ | CH₃ |
| 123 | F | H | H | D | CD₃ | CD₃ |
| 124 | F | H | H | H | CD₃ | CD₃ |
| 125 | F | D | D | D | CD₃ | CD₃ |
| 126 | F | D | D | D | CH₃ | CH₃ |
| 127 | F | D | D | H | CH₃ | CH₃ |
| 128 | F | D | D | H | CD₃ | CD₃ |
| 129 | N(CH₃)₂ | H | H | D | CH₃ | CH₃ |
| 130 | N(CH₃)₂ | H | H | D | CD₃ | CD₃ |
| 131 | N(CH₃)₂ | H | H | H | CD₃ | CD₃ |
| 132 | N(CH₃)₂ | D | D | D | CD₃ | CD₃ |
| 133 | N(CH₃)₂ | D | D | D | CH₃ | CH₃ |
| 134 | N(CH₃)₂ | D | D | H | CH₃ | CH₃ |
| 135 | N(CH₃)₂ | D | D | H | CD₃ | CD₃ |
| 136 | N(CD₃)₂ | H | H | D | CH₃ | CH₃ |
| 137 | N(CD₃)₂ | H | H | D | CD₃ | CD₃ |
| 138 | N(CD₃)₂ | H | H | H | CD₃ | CD₃ |
| 139 | N(CD₃)₂ | D | D | D | CD₃ | CD₃ |
| 140 | N(CD₃)₂ | D | D | D | CH₃ | CH₃ |
| 141 | N(CD₃)₂ | D | D | H | CH₃ | CH₃ |
| 142 | N(CD₃)₂ | D | D | H | CD₃ | CD₃ |
| 143 | N(CD₃)₂ | H | H | H | CH₃ | CH₃ |

Specific examples of a compound of Formula I include one of the compounds of Table 3 or a pharmaceutically acceptable salt thereof, wherein V is CH; each of X^{2a}, X^{2b}, X³ and X⁴ is hydrogen, and Z is NHCOCH₂NHCH₂CH₂CH₂CH₃:

TABLE 3

| Cmpd | W | X^{1a} | X^{1b} | X⁵ | Y¹ | Y² |
|---|---|---|---|---|---|---|
| 144 | F | H | H | D | CH₃ | CH₃ |
| 145 | F | H | H | D | CD₃ | CD₃ |
| 146 | F | H | H | H | CD₃ | CD₃ |
| 147 | F | D | D | D | CD₃ | CD₃ |
| 148 | F | D | D | D | CH₃ | CH₃ |
| 149 | F | D | D | H | CH₃ | CH₃ |
| 150 | F | D | D | H | CD₃ | CD₃ |
| 151 | N(CH₃)₂ | H | H | D | CH₃ | CH₃ |
| 152 | N(CH₃)₂ | H | H | D | CD₃ | CD₃ |
| 153 | N(CH₃)₂ | H | H | H | CD₃ | CD₃ |
| 154 | N(CH₃)₂ | D | D | D | CD₃ | CD₃ |
| 155 | N(CH₃)₂ | D | D | D | CH₃ | CH₃ |
| 156 | N(CH₃)₂ | D | D | H | CH₃ | CH₃ |
| 157 | N(CH₃)₂ | D | D | H | CD₃ | CD₃ |
| 158 | N(CD₃)₂ | H | H | D | CH₃ | CH₃ |
| 159 | N(CD₃)₂ | H | H | D | CD₃ | CD₃ |
| 160 | N(CD₃)₂ | H | H | H | CD₃ | CD₃ |
| 161 | N(CD₃)₂ | D | D | D | CD₃ | CD₃ |
| 162 | N(CD₃)₂ | D | D | D | CH₃ | CH₃ |
| 163 | N(CD₃)₂ | D | D | H | CH₃ | CH₃ |
| 164 | N(CD₃)₂ | D | D | H | CD₃ | CD₃ |
| 165 | N(CD₃)₂ | D | D | H | CH₃ | CH₃ |

Specific examples of a compound of Formula I include one of the compounds of Table 4 or a pharmaceutically acceptable salt thereof, wherein V is CH; each of X^{2a}, X^{2b}, X³ and X⁴ is hydrogen, and Z is

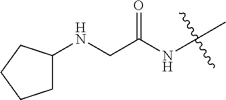 [SMILES: *NC(=O)CNC1CCCC1]

TABLE 4

| Cmpd | W | X^{1a} | X^{1b} | X⁵ | Y¹ | Y² |
|---|---|---|---|---|---|---|
| 166 | F | H | H | D | CH₃ | CH₃ |
| 167 | F | H | H | D | CD₃ | CD₃ |
| 168 | F | H | H | H | CD₃ | CD₃ |
| 169 | F | D | D | D | CD₃ | CD₃ |
| 170 | F | D | D | D | CH₃ | CH₃ |
| 171 | F | D | D | H | CH₃ | CH₃ |
| 172 | F | D | D | H | CD₃ | CD₃ |
| 173 | N(CH₃)₂ | H | H | D | CH₃ | CH₃ |
| 174 | N(CH₃)₂ | H | H | D | CD₃ | CD₃ |
| 175 | N(CH₃)₂ | H | H | H | CD₃ | CD₃ |
| 176 | N(CH₃)₂ | D | D | D | CD₃ | CD₃ |
| 177 | N(CH₃)₂ | D | D | D | CH₃ | CH₃ |
| 178 | N(CH₃)₂ | D | D | H | CH₃ | CH₃ |
| 179 | N(CH₃)₂ | D | D | H | CD₃ | CD₃ |
| 180 | N(CD₃)₂ | H | H | D | CH₃ | CH₃ |
| 181 | N(CD₃)₂ | H | H | D | CD₃ | CD₃ |
| 182 | N(CD₃)₂ | H | H | H | CD₃ | CD₃ |
| 183 | N(CD₃)₂ | D | D | D | CD₃ | CD₃ |
| 184 | N(CD₃)₂ | D | D | D | CH₃ | CH₃ |
| 185 | N(CD₃)₂ | D | D | H | CH₃ | CH₃ |
| 186 | N(CD₃)₂ | D | D | H | CD₃ | CD₃ |
| 187 | N(CD₃)₂ | H | H | H | CH₃ | CH₃ |

Specific examples of a compound of Formula I include one of the compounds of Table 5 or a pharmaceutically acceptable salt thereof, wherein V is CH; each of X^{2a}, X^{2b}, X³ and X⁴ is hydrogen, and Z is

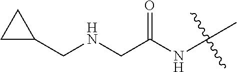 [SMILES: *NC(=O)CNCC1CC1]

TABLE 5

| Cmpd | W | X^{1a} | X^{1b} | X⁵ | Y¹ | Y² |
|---|---|---|---|---|---|---|
| 188 | F | H | H | D | CH₃ | CH₃ |
| 189 | F | H | H | D | CD₃ | CD₃ |
| 190 | F | H | H | H | CD₃ | CD₃ |
| 191 | F | D | D | D | CD₃ | CD₃ |
| 192 | F | D | D | D | CH₃ | CH₃ |
| 193 | F | D | D | H | CH₃ | CH₃ |
| 194 | F | D | D | H | CD₃ | CD₃ |
| 195 | N(CH₃)₂ | H | H | D | CH₃ | CH₃ |
| 196 | N(CH₃)₂ | H | H | D | CD₃ | CD₃ |
| 197 | N(CH₃)₂ | H | H | H | CD₃ | CD₃ |
| 198 | N(CH₃)₂ | D | D | D | CD₃ | CD₃ |
| 199 | N(CH₃)₂ | D | D | D | CH₃ | CH₃ |
| 200 | N(CH₃)₂ | D | D | H | CH₃ | CH₃ |
| 201 | N(CH₃)₂ | D | D | H | CD₃ | CD₃ |
| 202 | N(CD₃)₂ | H | H | D | CH₃ | CH₃ |
| 203 | N(CD₃)₂ | H | H | D | CD₃ | CD₃ |
| 204 | N(CD₃)₂ | H | H | H | CD₃ | CD₃ |
| 205 | N(CD₃)₂ | D | D | D | CD₃ | CD₃ |
| 206 | N(CD₃)₂ | D | D | D | CH₃ | CH₃ |
| 207 | N(CD₃)₂ | D | D | H | CH₃ | CH₃ |
| 208 | N(CD₃)₂ | D | D | H | CD₃ | CD₃ |
| 209 | N(CD₃)₂ | H | H | H | CH₃ | CH₃ |

Specific examples of a compound of Formula I include one of the compounds of Table 6 or a pharmaceutically acceptable salt thereof, wherein V is CH; each of X^{2a}, X^{2b}, X³ and X⁴ is hydrogen, and Z is

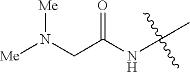 [SMILES: *NC(=O)CN(C)C]

TABLE 6

| Cmpd | W | X^{1a} | X^{1b} | X⁵ | Y¹ | Y² |
|---|---|---|---|---|---|---|
| 210 | F | H | H | D | CH₃ | CH₃ |
| 211 | F | H | H | D | CD₃ | CD₃ |
| 212 | F | H | H | H | CD₃ | CD₃ |
| 213 | F | D | D | D | CD₃ | CD₃ |
| 214 | F | D | D | D | CH₃ | CH₃ |
| 215 | F | D | D | H | CH₃ | CH₃ |
| 216 | F | D | D | H | CD₃ | CD₃ |
| 217 | N(CH₃)₂ | H | H | D | CH₃ | CH₃ |
| 218 | N(CH₃)₂ | H | H | D | CD₃ | CD₃ |
| 219 | N(CH₃)₂ | H | H | H | CD₃ | CD₃ |
| 220 | N(CH₃)₂ | D | D | D | CD₃ | CD₃ |
| 221 | N(CH₃)₂ | D | D | D | CH₃ | CH₃ |
| 222 | N(CH₃)₂ | D | D | H | CH₃ | CH₃ |
| 223 | N(CH₃)₂ | D | D | H | CD₃ | CD₃ |
| 224 | N(CD₃)₂ | H | H | D | CH₃ | CH₃ |
| 225 | N(CD₃)₂ | H | H | D | CD₃ | CD₃ |
| 226 | N(CD₃)₂ | H | H | H | CD₃ | CD₃ |
| 227 | N(CD₃)₂ | D | D | D | CD₃ | CD₃ |
| 228 | N(CD₃)₂ | D | D | D | CH₃ | CH₃ |
| 229 | N(CD₃)₂ | D | D | H | CH₃ | CH₃ |
| 230 | N(CD₃)₂ | D | D | H | CD₃ | CD₃ |
| 231 | N(CD₃)₂ | H | H | H | CH₃ | CH₃ |

Specific examples of a compound of Formula I include one of the compounds of Table 7 or a pharmaceutically acceptable salt thereof, wherein V is CH; each of X^{2a}, X^{2b}, X³ and X⁴ is hydrogen, and Z is

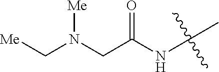 [SMILES: *NC(=O)CN(C)CC]

TABLE 7

| Cmpd | W | X^{1a} | X^{1b} | X⁵ | Y¹ | Y² |
|---|---|---|---|---|---|---|
| 232 | F | H | H | D | CH₃ | CH₃ |
| 233 | F | H | H | D | CD₃ | CD₃ |
| 234 | F | H | H | H | CD₃ | CD₃ |
| 235 | F | D | D | D | CD₃ | CD₃ |
| 236 | F | D | D | D | CH₃ | CH₃ |
| 237 | F | D | D | H | CH₃ | CH₃ |
| 238 | F | D | D | H | CD₃ | CD₃ |
| 239 | N(CH₃)₂ | H | H | D | CH₃ | CH₃ |
| 240 | N(CH₃)₂ | H | H | D | CD₃ | CD₃ |
| 241 | N(CH₃)₂ | H | H | H | CD₃ | CD₃ |
| 242 | N(CH₃)₂ | D | D | D | CD₃ | CD₃ |
| 243 | N(CH₃)₂ | D | D | D | CH₃ | CH₃ |
| 244 | N(CH₃)₂ | D | D | H | CH₃ | CH₃ |
| 245 | N(CH₃)₂ | D | D | H | CD₃ | CD₃ |
| 246 | N(CD₃)₂ | H | H | D | CH₃ | CH₃ |
| 247 | N(CD₃)₂ | H | H | D | CD₃ | CD₃ |
| 248 | N(CD₃)₂ | H | H | H | CD₃ | CD₃ |
| 249 | N(CD₃)₂ | D | D | D | CD₃ | CD₃ |
| 250 | N(CD₃)₂ | D | D | D | CH₃ | CH₃ |
| 251 | N(CD₃)₂ | D | D | H | CH₃ | CH₃ |
| 252 | N(CD₃)₂ | D | D | H | CD₃ | CD₃ |
| 253 | N(CD₃)₂ | H | H | H | CH₃ | CH₃ |

Specific examples of a compound of Formula I include one of the compounds of Table 8a or a pharmaceutically acceptable salt thereof, wherein V is CH; each of X^{2a}, X^{2b}, X³ and X⁴ is hydrogen, and Z is

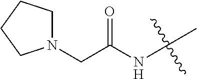 [SMILES: *NC(=O)CN1CCCC1]

TABLE 8a

| Cmpd | W | X^{1a} | X^{1b} | X⁵ | Y¹ | Y² |
|---|---|---|---|---|---|---|
| 254a | F | H | H | D | CH₃ | CH₃ |
| 255a | F | H | H | D | CD₃ | CD₃ |
| 256a | F | H | H | H | CD₃ | CD₃ |
| 257a | F | D | D | D | CD₃ | CD₃ |
| 258a | F | D | D | D | CH₃ | CH₃ |
| 259a | F | D | D | H | CH₃ | CH₃ |
| 260a | F | D | D | H | CD₃ | CD₃ |
| 261a | N(CH₃)₂ | H | H | D | CH₃ | CH₃ |
| 262a | N(CH₃)₂ | H | H | D | CD₃ | CD₃ |
| 263a | N(CH₃)₂ | H | H | H | CD₃ | CD₃ |
| 264a | N(CH₃)₂ | D | D | D | CD₃ | CD₃ |
| 265a | N(CH₃)₂ | D | D | D | CH₃ | CH₃ |
| 266a | N(CH₃)₂ | D | D | H | CH₃ | CH₃ |
| 267a | N(CH₃)₂ | D | D | H | CD₃ | CD₃ |
| 268a | N(CD₃)₂ | H | H | D | CH₃ | CH₃ |
| 269a | N(CD₃)₂ | H | H | D | CD₃ | CD₃ |
| 270a | N(CD₃)₂ | H | H | H | CD₃ | CD₃ |
| 271a | N(CD₃)₂ | D | D | D | CD₃ | CD₃ |
| 272a | N(CD₃)₂ | D | D | D | CH₃ | CH₃ |
| 273a | N(CD₃)₂ | D | D | H | CH₃ | CH₃ |
| 274a | N(CD₃)₂ | D | D | H | CD₃ | CD₃ |
| 275a | N(CD₃)₂ | H | H | H | CH₃ | CH₃ |

Specific examples of a compound of Formula I include one of the compounds of Table 8b or a pharmaceutically acceptable salt thereof, wherein V is CH; each of X^{2a}, X^{2b}, X³ and X⁴ is hydrogen, and Z is

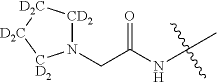

TABLE 8b

| Cmpd | W | X^{1a} | X^{1b} | X⁵ | Y¹ | Y² |
|---|---|---|---|---|---|---|
| 254b | F | H | H | D | CH₃ | CH₃ |
| 255b | F | H | H | D | CD₃ | CD₃ |
| 256b | F | H | H | H | CD₃ | CD₃ |
| 257b | F | D | D | D | CD₃ | CD₃ |
| 258b | F | D | D | D | CH₃ | CH₃ |
| 259b | F | D | D | H | CH₃ | CH₃ |
| 260b | F | D | D | H | CD₃ | CD₃ |
| 261b | N(CH₃)₂ | H | H | D | CH₃ | CH₃ |
| 262b | N(CH₃)₂ | H | H | D | CD₃ | CD₃ |
| 263b | N(CH₃)₂ | H | H | H | CD₃ | CD₃ |
| 264b | N(CH₃)₂ | D | D | D | CD₃ | CD₃ |
| 265b | N(CH₃)₂ | D | D | D | CH₃ | CH₃ |
| 266b | N(CH₃)₂ | D | D | H | CH₃ | CH₃ |
| 267b | N(CH₃)₂ | D | D | H | CD₃ | CD₃ |
| 268b | N(CD₃)₂ | H | H | D | CH₃ | CH₃ |
| 269b | N(CD₃)₂ | H | H | D | CD₃ | CD₃ |
| 270b | N(CD₃)₂ | H | H | H | CD₃ | CD₃ |
| 271b | N(CD₃)₂ | D | D | D | CD₃ | CD₃ |
| 272b | N(CD₃)₂ | D | D | D | CH₃ | CH₃ |
| 273b | N(CD₃)₂ | D | D | H | CH₃ | CH₃ |
| 274b | N(CD₃)₂ | D | D | H | CD₃ | CD₃ |
| 275b | N(CD₃)₂ | H | H | H | CH₃ | CH₃ |

Specific examples of a compound of Formula I include one of the compounds of Table 8c or a pharmaceutically acceptable salt thereof, wherein V is CH; each of X^{2a}, X^{2b}, X³ and X⁴ is hydrogen, and Z is

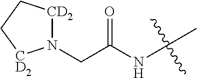

TABLE 8c

| Cmpd | W | X^{1a} | X^{1b} | X⁵ | Y¹ | Y² |
|---|---|---|---|---|---|---|
| 254c | F | H | H | D | CH₃ | CH₃ |
| 255c | F | H | H | D | CD₃ | CD₃ |
| 256c | F | H | H | H | CD₃ | CD₃ |
| 257c | F | D | D | D | CD₃ | CD₃ |
| 258c | F | D | D | D | CH₃ | CH₃ |
| 259c | F | D | D | H | CH₃ | CH₃ |
| 260c | F | D | D | H | CD₃ | CD₃ |
| 261c | N(CH₃)₂ | H | H | D | CH₃ | CH₃ |
| 262c | N(CH₃)₂ | H | H | D | CD₃ | CD₃ |
| 263c | N(CH₃)₂ | H | H | H | CD₃ | CD₃ |
| 264c | N(CH₃)₂ | D | D | D | CD₃ | CD₃ |
| 265c | N(CH₃)₂ | D | D | D | CH₃ | CH₃ |
| 266c | N(CH₃)₂ | D | D | H | CH₃ | CH₃ |
| 267c | N(CH₃)₂ | D | D | H | CD₃ | CD₃ |
| 268c | N(CD₃)₂ | H | H | D | CH₃ | CH₃ |
| 269c | N(CD₃)₂ | H | H | D | CD₃ | CD₃ |
| 270c | N(CD₃)₂ | H | H | H | CD₃ | CD₃ |
| 271c | N(CD₃)₂ | D | D | D | CD₃ | CD₃ |
| 272c | N(CD₃)₂ | D | D | D | CH₃ | CH₃ |
| 273c | N(CD₃)₂ | D | D | H | CH₃ | CH₃ |
| 274c | N(CD₃)₂ | D | D | H | CD₃ | CD₃ |
| 275c | N(CD₃)₂ | H | H | H | CH₃ | CH₃ |

Specific examples of a compound of Formula I include one of the compounds of Table 8d or a pharmaceutically acceptable salt thereof, wherein V is CH; each of X^{2a}, X^{2b}, X³ and X⁴ is hydrogen, and Z is

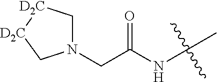

TABLE 8d

| Cmpd | W | X^{1a} | X^{1b} | X⁵ | Y¹ | Y² |
|---|---|---|---|---|---|---|
| 254d | F | H | H | D | CH₃ | CH₃ |
| 255d | F | H | H | D | CD₃ | CD₃ |
| 256d | F | H | H | H | CD₃ | CD₃ |
| 257d | F | D | D | D | CD₃ | CD₃ |
| 258d | F | D | D | D | CH₃ | CH₃ |
| 259d | F | D | D | H | CH₃ | CH₃ |
| 260d | F | D | D | H | CD₃ | CD₃ |
| 261d | N(CH₃)₂ | H | H | D | CH₃ | CH₃ |
| 262d | N(CH₃)₂ | H | H | D | CD₃ | CD₃ |
| 263d | N(CH₃)₂ | H | H | H | CD₃ | CD₃ |
| 264d | N(CH₃)₂ | D | D | D | CD₃ | CD₃ |
| 265d | N(CH₃)₂ | D | D | D | CH₃ | CH₃ |
| 266d | N(CH₃)₂ | D | D | H | CH₃ | CH₃ |
| 267d | N(CH₃)₂ | D | D | H | CD₃ | CD₃ |
| 268d | N(CD₃)₂ | H | H | D | CH₃ | CH₃ |
| 269d | N(CD₃)₂ | H | H | D | CD₃ | CD₃ |
| 270d | N(CD₃)₂ | H | H | H | CD₃ | CD₃ |
| 271d | N(CD₃)₂ | D | D | D | CD₃ | CD₃ |
| 272d | N(CD₃)₂ | D | D | D | CH₃ | CH₃ |
| 273d | N(CD₃)₂ | D | D | H | CH₃ | CH₃ |
| 274d | N(CD₃)₂ | D | D | H | CD₃ | CD₃ |
| 275d | N(CD₃)₂ | H | H | H | CH₃ | CH₃ |

Specific examples of a compound of Formula I include one of the compounds of Table 8e or a pharmaceutically acceptable salt thereof, wherein V is CH; each of X^{2a}, X^{2b}, X³ and X⁴ is hydrogen, and Z is

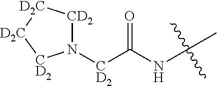

TABLE 8e

| Cmpd | W | X^{1a} | X^{1b} | X⁵ | Y¹ | Y² |
|---|---|---|---|---|---|---|
| 254e | F | H | H | D | CH₃ | CH₃ |
| 255e | F | H | H | D | CD₃ | CD₃ |
| 256e | F | H | H | H | CD₃ | CD₃ |
| 257e | F | D | D | D | CD₃ | CD₃ |
| 258e | F | D | D | D | CH₃ | CH₃ |
| 259e | F | D | D | H | CH₃ | CH₃ |
| 260e | F | D | D | H | CD₃ | CD₃ |
| 261e | N(CH₃)₂ | H | H | D | CH₃ | CH₃ |
| 262e | N(CH₃)₂ | H | H | D | CD₃ | CD₃ |
| 263e | N(CH₃)₂ | H | H | H | CD₃ | CD₃ |
| 264e | N(CH₃)₂ | D | D | D | CD₃ | CD₃ |
| 265e | N(CH₃)₂ | D | D | D | CH₃ | CH₃ |
| 266e | N(CH₃)₂ | D | D | H | CH₃ | CH₃ |
| 267e | N(CH₃)₂ | D | D | H | CD₃ | CD₃ |
| 268e | N(CD₃)₂ | H | H | D | CH₃ | CH₃ |
| 269e | N(CD₃)₂ | H | H | D | CD₃ | CD₃ |
| 270e | N(CD₃)₂ | H | H | H | CD₃ | CD₃ |
| 271e | N(CD₃)₂ | D | D | D | CD₃ | CD₃ |
| 272e | N(CD₃)₂ | D | D | D | CH₃ | CH₃ |
| 273e | N(CD₃)₂ | D | D | H | CH₃ | CH₃ |
| 274e | N(CD₃)₂ | D | D | H | CD₃ | CD₃ |
| 275e | N(CD₃)₂ | H | H | H | CH₃ | CH₃ |

Specific examples of a compound of Formula I include one of the compounds of Table 8f or a pharmaceutically acceptable salt thereof, wherein V is CH; each of X^{2a}, X^{2b}, X³ and X⁴ is hydrogen, and Z is

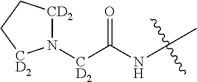

TABLE 8f

| Cmpd | W | X^{1a} | X^{1b} | X⁵ | Y¹ | Y² |
|---|---|---|---|---|---|---|
| 254f | F | H | H | D | CH₃ | CH₃ |
| 255f | F | H | H | D | CD₃ | CD₃ |
| 256f | F | H | H | H | CD₃ | CD₃ |
| 257f | F | D | D | D | CD₃ | CD₃ |
| 258f | F | D | D | D | CH₃ | CH₃ |
| 259f | F | D | D | H | CH₃ | CH₃ |
| 260f | F | D | D | H | CD₃ | CD₃ |
| 261f | N(CH₃)₂ | D | H | D | CH₃ | CH₃ |
| 262f | N(CH₃)₂ | D | H | D | CD₃ | CD₃ |
| 263f | N(CH₃)₂ | D | H | H | CD₃ | CD₃ |
| 264f | N(CH₃)₂ | D | D | D | CD₃ | CD₃ |
| 265f | N(CH₃)₂ | D | D | D | CH₃ | CH₃ |
| 266f | N(CH₃)₂ | D | D | H | CH₃ | CH₃ |
| 267f | N(CH₃)₂ | D | D | H | CD₃ | CD₃ |
| 268f | N(CD₃)₂ | H | H | D | CH₃ | CH₃ |
| 269f | N(CD₃)₂ | H | H | D | CD₃ | CD₃ |
| 270f | N(CD₃)₂ | H | H | H | CD₃ | CD₃ |
| 271f | N(CD₃)₂ | D | D | D | CD₃ | CD₃ |
| 272f | N(CD₃)₂ | D | D | D | CH₃ | CH₃ |
| 273f | N(CD₃)₂ | D | D | H | CH₃ | CH₃ |
| 274f | N(CD₃)₂ | D | D | H | CD₃ | CD₃ |
| 275f | N(CD₃)₂ | H | H | H | CH₃ | CH₃ |

Specific examples of a compound of Formula I include one of the compounds of Table 8g or a pharmaceutically acceptable salt thereof, wherein V is CH; each of X^{2a}, X^{2b}, X³ and X⁴ is hydrogen, and Z is

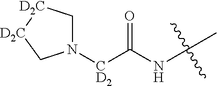

TABLE 8g

| Cmpd | W | X^{1a} | X^{1b} | X⁵ | Y¹ | Y² |
|---|---|---|---|---|---|---|
| 254g | F | H | H | D | CH₃ | CH₃ |
| 255g | F | H | H | D | CD₃ | CD₃ |
| 256g | F | H | H | H | CD₃ | CD₃ |
| 257g | F | D | D | D | CD₃ | CD₃ |
| 258g | F | D | D | D | CH₃ | CH₃ |
| 259g | F | D | D | H | CH₃ | CH₃ |
| 260g | F | D | D | H | CD₃ | CD₃ |
| 261g | N(CH₃)₂ | H | H | D | CH₃ | CH₃ |
| 262g | N(CH₃)₂ | H | H | D | CD₃ | CD₃ |
| 263g | N(CH₃)₂ | H | H | H | CD₃ | CD₃ |
| 264g | N(CH₃)₂ | D | D | D | CD₃ | CD₃ |
| 265g | N(CH₃)₂ | D | D | D | CH₃ | CH₃ |
| 266g | N(CH₃)₂ | D | D | H | CH₃ | CH₃ |
| 267g | N(CH₃)₂ | D | D | H | CD₃ | CD₃ |
| 268g | N(CD₃)₂ | H | H | D | CH₃ | CH₃ |
| 269g | N(CD₃)₂ | H | H | D | CD₃ | CD₃ |
| 270g | N(CD₃)₂ | H | H | H | CD₃ | CD₃ |
| 271g | N(CD₃)₂ | D | D | D | CD₃ | CD₃ |
| 272g | N(CD₃)₂ | D | D | D | CH₃ | CH₃ |
| 273g | N(CD₃)₂ | D | D | H | CH₃ | CH₃ |
| 274g | N(CD₃)₂ | D | D | H | CD₃ | CD₃ |
| 275g | N(CD₃)₂ | H | H | H | CH₃ | CH₃ |

Specific examples of a compound of Formula I include one of the compounds of Table 9 or a pharmaceutically acceptable salt thereof, wherein V is CH; each of X^{2a}, X^{2b}, X³ and X⁴ is hydrogen, and Z is

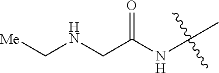 [SMILES: *NC(=O)CNCC]

TABLE 9

| Cmpd | W | X^{1a} | X^{1b} | X⁵ | Y¹ | Y² |
|---|---|---|---|---|---|---|
| 276 | F | H | H | D | CH₃ | CH₃ |
| 277 | F | H | H | D | CD₃ | CD₃ |
| 278 | F | H | H | H | CD₃ | CD₃ |
| 279 | F | D | D | D | CD₃ | CD₃ |
| 280 | F | D | D | D | CH₃ | CH₃ |
| 281 | F | D | D | H | CH₃ | CH₃ |
| 282 | F | D | D | H | CD₃ | CD₃ |
| 283 | N(CH₃)₂ | H | H | D | CH₃ | CH₃ |
| 284 | N(CH₃)₂ | H | H | D | CD₃ | CD₃ |
| 285 | N(CH₃)₂ | H | H | H | CD₃ | CD₃ |
| 286 | N(CH₃)₂ | D | D | D | CD₃ | CD₃ |
| 287 | N(CH₃)₂ | D | D | D | CH₃ | CH₃ |
| 288 | N(CH₃)₂ | D | D | H | CH₃ | CH₃ |
| 289 | N(CH₃)₂ | D | D | H | CD₃ | CD₃ |
| 290 | N(CD₃)₂ | H | H | D | CH₃ | CH₃ |
| 291 | N(CD₃)₂ | H | H | D | CD₃ | CD₃ |
| 292 | N(CD₃)₂ | H | H | H | CD₃ | CD₃ |
| 293 | N(CD₃)₂ | D | D | D | CD₃ | CD₃ |
| 294 | N(CD₃)₂ | D | D | D | CH₃ | CH₃ |
| 295 | N(CD₃)₂ | D | D | H | CH₃ | CH₃ |
| 296 | N(CD₃)₂ | D | D | H | CD₃ | CD₃ |
| 297 | N(CD₃)₂ | H | H | H | CH₃ | CH₃ |

Specific examples of a compound of Formula I include one of the compounds of Table 10 or a pharmaceutically acceptable salt thereof, wherein V is CH; each of X^{2a}, X^{2b}, X³ and X⁴ is hydrogen, and Z is

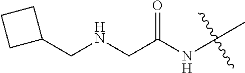 [SMILES: *NC(=O)CNCC1CCC1]

TABLE 10

| Cmpd | W | X^{1a} | X^{1b} | X⁵ | Y¹ | Y² |
|---|---|---|---|---|---|---|
| 298 | F | H | H | D | CH₃ | CH₃ |
| 299 | F | H | H | D | CD₃ | CD₃ |
| 300 | F | H | H | H | CD₃ | CD₃ |
| 301 | F | D | D | D | CD₃ | CD₃ |
| 302 | F | D | D | D | CH₃ | CH₃ |
| 303 | F | D | D | H | CH₃ | CH₃ |
| 304 | F | D | D | H | CD₃ | CD₃ |
| 305 | N(CH₃)₂ | H | H | D | CH₃ | CH₃ |
| 306 | N(CH₃)₂ | H | H | D | CD₃ | CD₃ |
| 307 | N(CH₃)₂ | H | H | H | CD₃ | CD₃ |
| 308 | N(CH₃)₂ | D | D | D | CD₃ | CD₃ |
| 309 | N(CH₃)₂ | D | D | D | CH₃ | CH₃ |
| 310 | N(CH₃)₂ | D | D | H | CH₃ | CH₃ |
| 311 | N(CH₃)₂ | D | D | H | CD₃ | CD₃ |
| 312 | N(CD₃)₂ | H | H | D | CH₃ | CH₃ |
| 313 | N(CD₃)₂ | H | H | D | CD₃ | CD₃ |
| 314 | N(CD₃)₂ | H | H | H | CD₃ | CD₃ |
| 315 | N(CD₃)₂ | D | D | D | CD₃ | CD₃ |
| 316 | N(CD₃)₂ | D | D | D | CH₃ | CH₃ |
| 317 | N(CD₃)₂ | D | D | H | CH₃ | CH₃ |
| 318 | N(CD₃)₂ | D | D | H | CD₃ | CD₃ |
| 319 | N(CD₃)₂ | H | H | H | CH₃ | CH₃ |

Specific examples of a compound of Formula I include one of the compounds of Table 11 or a pharmaceutically acceptable salt thereof, wherein V is CH; each of X^{2a}, X^{2b}, X³ and X⁴ is hydrogen, and Z is

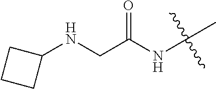 [SMILES: *NC(=O)CNC1CCC1]

TABLE 11

| Cmpd | W | X^{1a} | X^{1b} | X⁵ | Y¹ | Y² |
|---|---|---|---|---|---|---|
| 320 | F | H | H | D | CH₃ | CH₃ |
| 321 | F | H | H | D | CD₃ | CD₃ |
| 322 | F | H | H | H | CD₃ | CD₃ |
| 323 | F | D | D | D | CD₃ | CD₃ |
| 324 | F | D | D | D | CH₃ | CH₃ |
| 325 | F | D | D | H | CH₃ | CH₃ |
| 326 | F | D | D | H | CD₃ | CD₃ |
| 327 | N(CH₃)₂ | H | H | D | CH₃ | CH₃ |
| 328 | N(CH₃)₂ | H | H | D | CD₃ | CD₃ |
| 329 | N(CH₃)₂ | H | H | H | CD₃ | CD₃ |
| 330 | N(CH₃)₂ | D | D | D | CD₃ | CD₃ |
| 331 | N(CH₃)₂ | D | D | D | CH₃ | CH₃ |
| 332 | N(CH₃)₂ | D | D | H | CH₃ | CH₃ |
| 333 | N(CH₃)₂ | D | D | H | CD₃ | CD₃ |
| 334 | N(CD₃)₂ | H | H | D | CH₃ | CH₃ |
| 335 | N(CD₃)₂ | H | H | D | CD₃ | CD₃ |
| 336 | N(CD₃)₂ | H | H | H | CD₃ | CD₃ |
| 337 | N(CD₃)₂ | D | D | D | CD₃ | CD₃ |
| 338 | N(CD₃)₂ | D | D | D | CH₃ | CH₃ |
| 339 | N(CD₃)₂ | D | D | H | CH₃ | CH₃ |
| 340 | N(CD₃)₂ | D | D | H | CD₃ | CD₃ |
| 341 | N(CD₃)₂ | H | H | H | CH₃ | CH₃ |

Specific examples of a compound of Formula I include one of the compounds of Table 12 or a pharmaceutically acceptable salt thereof, wherein V is CH; each of X^{2a}, X^{2b}, X³ and X⁴ is hydrogen, and Z is

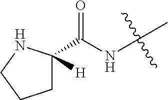 [SMILES: *NC(=O)[C@@H]1CCCN1]

TABLE 12

| Cmpd | W | X^{1a} | X^{1b} | X⁵ | Y¹ | Y² |
|---|---|---|---|---|---|---|
| 342 | F | H | H | D | CH₃ | CH₃ |
| 343 | F | H | H | D | CD₃ | CD₃ |
| 344 | F | H | H | H | CD₃ | CD₃ |
| 345 | F | D | D | D | CD₃ | CD₃ |
| 346 | F | D | D | D | CH₃ | CH₃ |
| 347 | F | D | D | H | CH₃ | CH₃ |
| 348 | F | D | D | H | CD₃ | CD₃ |
| 349 | N(CH₃)₂ | H | H | D | CH₃ | CH₃ |
| 350 | N(CH₃)₂ | H | H | D | CD₃ | CD₃ |
| 351 | N(CH₃)₂ | H | H | H | CD₃ | CD₃ |
| 352 | N(CH₃)₂ | D | D | D | CD₃ | CD₃ |
| 353 | N(CH₃)₂ | D | D | D | CH₃ | CH₃ |
| 354 | N(CH₃)₂ | D | D | H | CH₃ | CH₃ |
| 355 | N(CH₃)₂ | D | D | H | CD₃ | CD₃ |
| 356 | N(CD₃)₂ | H | H | D | CH₃ | CH₃ |
| 357 | N(CD₃)₂ | H | H | D | CD₃ | CD₃ |
| 358 | N(CD₃)₂ | H | H | H | CD₃ | CD₃ |
| 359 | N(CD₃)₂ | D | D | D | CD₃ | CD₃ |
| 360 | N(CD₃)₂ | D | D | D | CH₃ | CH₃ |
| 361 | N(CD₃)₂ | D | D | H | CH₃ | CH₃ |
| 362 | N(CD₃)₂ | D | D | H | CD₃ | CD₃ |
| 363 | N(CD₃)₂ | H | H | H | CH₃ | CH₃ |

Specific examples of a compound of Formula I include one of the compounds of Table 13 or a pharmaceutically acceptable salt thereof, wherein V is CH; each of X^{2a}, X^{2b}, X³ and X⁴ is hydrogen, and Z is

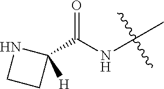 [SMILES: *NC(=O)[C@@H]1CCN1]

TABLE 13

| Cmpd | W | X^{1a} | X^{1b} | X⁵ | Y¹ | Y² |
|---|---|---|---|---|---|---|
| 364 | F | H | H | D | CH₃ | CH₃ |
| 365 | F | H | H | D | CD₃ | CD₃ |
| 366 | F | H | H | H | CD₃ | CD₃ |
| 367 | F | D | D | D | CD₃ | CD₃ |
| 368 | F | D | D | D | CH₃ | CH₃ |
| 369 | F | D | D | H | CH₃ | CH₃ |
| 370 | F | D | D | H | CD₃ | CD₃ |
| 371 | N(CH₃)₂ | H | H | D | CH₃ | CH₃ |
| 372 | N(CH₃)₂ | H | H | D | CD₃ | CD₃ |
| 373 | N(CH₃)₂ | H | H | H | CD₃ | CD₃ |
| 374 | N(CH₃)₂ | D | D | D | CD₃ | CD₃ |
| 375 | N(CH₃)₂ | D | D | D | CH₃ | CH₃ |
| 376 | N(CH₃)₂ | D | D | H | CH₃ | CH₃ |
| 377 | N(CH₃)₂ | D | D | H | CD₃ | CD₃ |
| 378 | N(CD₃)₂ | H | H | D | CH₃ | CH₃ |
| 379 | N(CD₃)₂ | H | H | D | CD₃ | CD₃ |
| 380 | N(CD₃)₂ | H | H | H | CD₃ | CD₃ |
| 381 | N(CD₃)₂ | D | D | D | CD₃ | CD₃ |
| 382 | N(CD₃)₂ | D | D | D | CH₃ | CH₃ |
| 383 | N(CD₃)₂ | D | D | H | CH₃ | CH₃ |
| 384 | N(CD₃)₂ | D | D | H | CD₃ | CD₃ |
| 385 | N(CD₃)₂ | H | H | H | CH₃ | CH₃ |

Specific examples of a compound of Formula I include one of the compounds of Table 14a or a pharmaceutically acceptable salt thereof, wherein V is CH; each of X^{2a}, X^{2b}, X³ and X⁴ is hydrogen, and Z is

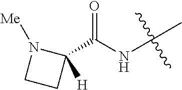 [SMILES: *NC(=O)[C@@H]1CCN1C]

TABLE 14a

| Cmpd | W | X^{1a} | X^{1b} | X⁵ | Y¹ | Y² |
|---|---|---|---|---|---|---|
| 386a | F | H | H | D | CH₃ | CH₃ |
| 387a | F | H | H | D | CD₃ | CD₃ |
| 388a | F | H | H | H | CD₃ | CD₃ |
| 389a | F | D | D | D | CD₃ | CD₃ |
| 390a | F | D | D | D | CH₃ | CH₃ |
| 391a | F | D | D | H | CH₃ | CH₃ |
| 392a | F | D | D | H | CD₃ | CD₃ |
| 393a | N(CH₃)₂ | H | H | D | CH₃ | CH₃ |
| 394a | N(CH₃)₂ | H | H | D | CD₃ | CD₃ |
| 395a | N(CH₃)₂ | H | H | H | CD₃ | CD₃ |
| 396a | N(CH₃)₂ | D | D | D | CD₃ | CD₃ |
| 397a | N(CH₃)₂ | D | D | D | CH₃ | CH₃ |
| 398a | N(CH₃)₂ | D | D | H | CH₃ | CH₃ |
| 399a | N(CH₃)₂ | D | D | H | CD₃ | CD₃ |
| 400a | N(CD₃)₂ | H | H | D | CH₃ | CH₃ |
| 401a | N(CD₃)₂ | H | H | D | CD₃ | CD₃ |
| 402a | N(CD₃)₂ | H | H | H | CD₃ | CD₃ |
| 403a | N(CD₃)₂ | D | D | D | CD₃ | CD₃ |
| 404a | N(CD₃)₂ | D | D | D | CH₃ | CH₃ |
| 405a | N(CD₃)₂ | D | D | H | CH₃ | CH₃ |
| 406a | N(CD₃)₂ | D | D | H | CD₃ | CD₃ |
| 407a | N(CD₃)₂ | H | H | H | CH₃ | CH₃ |

Specific examples of a compound of Formula I include one of the compounds of Table 14b or a pharmaceutically acceptable salt thereof, wherein V is CH; each of X^{2a}, X^{2b}, X³ and X⁴ is hydrogen, and Z is

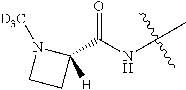 [SMILES: *NC(=O)[C@@H]1CCN1C([2H])([2H])[2H]]

TABLE 14b

| Cmpd | W | X^{1a} | X^{1b} | X⁵ | Y¹ | Y² |
|---|---|---|---|---|---|---|
| 386b | F | H | H | D | CH₃ | CH₃ |
| 387b | F | H | H | D | CD₃ | CD₃ |
| 388b | F | H | H | H | CD₃ | CD₃ |
| 389b | F | D | D | D | CD₃ | CD₃ |
| 390b | F | D | D | D | CH₃ | CH₃ |
| 391b | F | D | D | H | CH₃ | CH₃ |
| 392b | F | D | D | H | CD₃ | CD₃ |
| 393b | N(CH₃)₂ | H | H | D | CH₃ | CH₃ |
| 394b | N(CH₃)₂ | H | H | D | CD₃ | CD₃ |
| 395b | N(CH₃)₂ | H | H | H | CD₃ | CD₃ |
| 396b | N(CH₃)₂ | D | D | D | CD₃ | CD₃ |
| 397b | N(CH₃)₂ | D | D | D | CH₃ | CH₃ |
| 398b | N(CH₃)₂ | D | D | H | CH₃ | CH₃ |
| 399b | N(CH₃)₂ | D | D | H | CD₃ | CD₃ |
| 400b | N(CD₃)₂ | H | H | D | CH₃ | CH₃ |
| 401b | N(CD₃)₂ | H | H | D | CD₃ | CD₃ |
| 402b | N(CD₃)₂ | H | H | H | CD₃ | CD₃ |
| 403b | N(CD₃)₂ | D | D | D | CD₃ | CD₃ |
| 404b | N(CD₃)₂ | D | D | D | CH₃ | CH₃ |
| 405b | N(CD₃)₂ | D | D | H | CH₃ | CH₃ |
| 406b | N(CD₃)₂ | D | D | H | CD₃ | CD₃ |
| 407b | N(CD₃)₂ | H | H | H | CH₃ | CH₃ |

Specific examples of a compound of Formula I include one of the compounds of Table 15a or a pharmaceutically acceptable salt thereof, wherein V is CH; each of X^{2a}, X^{2b}, X³ and X⁴ is hydrogen, and Z is

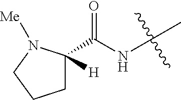 [SMILES: *NC(=O)[C@@H]1CCCN1C]

TABLE 15a

| Cmpd | W | X^{1a} | X^{1b} | X⁵ | Y¹ | Y² |
|---|---|---|---|---|---|---|
| 408a | F | H | H | H | CH₃ | CH₃ |
| 409a | F | H | H | D | CH₃ | CH₃ |
| 410a | F | H | H | D | CD₃ | CD₃ |
| 411a | F | H | H | H | CD₃ | CD₃ |
| 412a | F | D | D | D | CD₃ | CD₃ |
| 413a | F | D | D | D | CH₃ | CH₃ |
| 414a | F | D | D | H | CH₃ | CH₃ |
| 415a | F | D | D | H | CD₃ | CD₃ |
| 416a | N(CH₃)₂ | H | H | H | CH₃ | CH₃ |
| 417a | N(CH₃)₂ | H | H | D | CH₃ | CH₃ |
| 418a | N(CH₃)₂ | H | H | D | CD₃ | CD₃ |
| 419a | N(CH₃)₂ | H | H | H | CD₃ | CD₃ |
| 420a | N(CH₃)₂ | D | D | D | CD₃ | CD₃ |
| 421a | N(CH₃)₂ | D | D | D | CH₃ | CH₃ |
| 422a | N(CH₃)₂ | D | D | H | CH₃ | CH₃ |
| 423a | N(CH₃)₂ | D | D | H | CD₃ | CD₃ |
| 424a | N(CD₃)₂ | H | H | D | CH₃ | CH₃ |
| 425a | N(CD₃)₂ | H | H | D | CD₃ | CD₃ |
| 426a | N(CD₃)₂ | H | H | H | CD₃ | CD₃ |
| 427a | N(CD₃)₂ | D | D | D | CD₃ | CD₃ |
| 428a | N(CD₃)₂ | D | D | D | CH₃ | CH₃ |
| 429a | N(CD₃)₂ | D | D | H | CH₃ | CH₃ |
| 430a | N(CD₃)₂ | D | D | H | CD₃ | CD₃ |
| 431a | N(CD₃)₂ | H | H | H | CH₃ | CH₃ |

Specific examples of a compound of Formula I include one of the compounds of Table 15b or a pharmaceutically acceptable salt thereof, wherein V is CH; each of X^{2a}, X^{2b}, X³ and X⁴ is hydrogen, and Z is

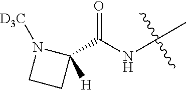 [SMILES: *NC(=O)[C@@H]1CCN1C([2H])([2H])[2H]]

TABLE 15b

| Cmpd | W | X^{1a} | X^{1b} | X⁵ | Y¹ | Y² |
|---|---|---|---|---|---|---|
| 408b | F | H | H | H | CH₃ | CH₃ |
| 409b | F | H | H | D | CH₃ | CH₃ |
| 410b | F | H | H | D | CD₃ | CD₃ |
| 411b | F | H | H | H | CD₃ | CD₃ |
| 412b | F | D | D | D | CD₃ | CD₃ |
| 413b | F | D | D | D | CH₃ | CH₃ |
| 414b | F | D | D | H | CH₃ | CH₃ |
| 415b | F | D | D | H | CD₃ | CD₃ |
| 416b | N(CH₃)₂ | H | H | H | CH₃ | CH₃ |
| 417b | N(CH₃)₂ | H | H | D | CH₃ | CH₃ |
| 418b | N(CH₃)₂ | H | H | D | CD₃ | CD₃ |
| 419b | N(CH₃)₂ | H | H | H | CD₃ | CD₃ |
| 420b | N(CH₃)₂ | D | D | D | CD₃ | CD₃ |
| 421b | N(CH₃)₂ | D | D | D | CH₃ | CH₃ |
| 422b | N(CH₃)₂ | D | D | H | CH₃ | CH₃ |
| 423b | N(CH₃)₂ | D | D | H | CD₃ | CD₃ |
| 424b | N(CD₃)₂ | H | H | D | CH₃ | CH₃ |
| 425b | N(CD₃)₂ | H | H | D | CD₃ | CD₃ |
| 426b | N(CD₃)₂ | H | H | H | CD₃ | CD₃ |
| 427b | N(CD₃)₂ | D | D | D | CD₃ | CD₃ |
| 428b | N(CD₃)₂ | D | D | D | CH₃ | CH₃ |
| 429b | N(CD₃)₂ | D | D | H | CH₃ | CH₃ |
| 430b | N(CD₃)₂ | D | D | H | CD₃ | CD₃ |
| 431b | N(CD₃)₂ | H | H | H | CH₃ | CH₃ |

Specific examples of a compound of Formula I include one of the compounds of Table 16 or a pharmaceutically acceptable salt thereof, wherein V is CH; each of X^{2a}, X^{2b}, X³ and X⁴ is hydrogen, and Z is CH₂NHCH₂C(CH₃)₃

TABLE 16

| Cmpd | W | X^{1a} | X^{1b} | X⁵ | Y¹ | Y² |
|---|---|---|---|---|---|---|
| 432 | F | H | H | D | CH₃ | CH₃ |
| 433 | F | H | H | D | CD₃ | CD₃ |
| 434 | F | H | H | H | CD₃ | CD₃ |
| 435 | F | D | D | D | CD₃ | CD₃ |
| 436 | F | D | D | D | CH₃ | CH₃ |
| 437 | F | D | D | H | CH₃ | CH₃ |
| 438 | F | D | D | H | CD₃ | CD₃ |
| 439 | N(CH₃)₂ | H | H | D | CH₃ | CH₃ |
| 440 | N(CH₃)₂ | H | H | D | CD₃ | CD₃ |
| 441 | N(CH₃)₂ | H | H | H | CD₃ | CD₃ |
| 442 | N(CH₃)₂ | D | D | D | CD₃ | CD₃ |
| 443 | N(CH₃)₂ | D | D | D | CH₃ | CH₃ |
| 444 | N(CH₃)₂ | D | D | H | CH₃ | CH₃ |
| 445 | N(CH₃)₂ | D | D | H | CD₃ | CD₃ |
| 446 | N(CD₃)₂ | H | H | D | CH₃ | CH₃ |
| 447 | N(CD₃)₂ | H | H | D | CD₃ | CD₃ |
| 448 | N(CD₃)₂ | H | H | H | CD₃ | CD₃ |
| 449 | N(CD₃)₂ | D | D | D | CD₃ | CD₃ |
| 450 | N(CD₃)₂ | D | D | D | CH₃ | CH₃ |
| 451 | N(CD₃)₂ | D | D | H | CH₃ | CH₃ |
| 452 | N(CD₃)₂ | D | D | H | CD₃ | CD₃ |

Specific examples of a compound of Formula I include one of the compounds of Table 17 or a pharmaceutically acceptable salt thereof, wherein V is CH; each of X^{2a}, X^{2b}, X³ and X⁴ is hydrogen, and Z is CD₂NHCD₂C(CD₃)₃

TABLE 17

| Cmpd | W | X^{1a} | X^{1b} | X⁵ | Y¹ | Y² |
|---|---|---|---|---|---|---|
| 453 | F | H | H | H | CH₃ | CH₃ |
| 454 | F | H | H | D | CH₃ | CH₃ |
| 455 | F | H | H | D | CD₃ | CD₃ |
| 456 | F | H | H | H | CD₃ | CD₃ |
| 457 | F | D | D | D | CD₃ | CD₃ |
| 458 | F | D | D | D | CH₃ | CH₃ |
| 459 | F | D | D | H | CH₃ | CH₃ |
| 460 | F | D | D | H | CD₃ | CD₃ |
| 461 | N(CH₃)₂ | H | H | H | CH₃ | CH₃ |
| 462 | N(CH₃)₂ | H | H | D | CH₃ | CH₃ |
| 463 | N(CH₃)₂ | H | H | D | CD₃ | CD₃ |
| 464 | N(CH₃)₂ | H | H | H | CD₃ | CD₃ |
| 465 | N(CH₃)₂ | D | D | D | CD₃ | CD₃ |
| 466 | N(CH₃)₂ | D | D | D | CH₃ | CH₃ |
| 467 | N(CH₃)₂ | D | D | H | CH₃ | CH₃ |
| 468 | N(CH₃)₂ | D | D | H | CD₃ | CD₃ |
| 469 | N(CD₃)₂ | H | H | D | CH₃ | CH₃ |
| 470 | N(CD₃)₂ | H | H | D | CD₃ | CD₃ |
| 471 | N(CD₃)₂ | H | H | H | CD₃ | CD₃ |
| 472 | N(CD₃)₂ | D | D | D | CD₃ | CD₃ |
| 473 | N(CD₃)₂ | D | D | D | CH₃ | CH₃ |
| 474 | N(CD₃)₂ | D | D | H | CH₃ | CH₃ |
| 475 | N(CD₃)₂ | D | D | H | CD₃ | CD₃ |
| 476 | N(CD₃)₂ | H | H | H | CH₃ | CH₃ |

Specific examples of a compound of Formula I include one of the compounds of Table 18 or a pharmaceutically acceptable salt thereof, wherein V is CH; each of X^{2a}, X^{2b}, X³ and X⁴ is hydrogen, and Z is NHC(O)CH₂NHC(CH₃)₃

TABLE 18

| Cmpd | W | X^{1a} | X^{1b} | X⁵ | Y¹ | Y² |
|---|---|---|---|---|---|---|
| 477 | F | H | H | D | CH₃ | CH₃ |
| 478 | F | H | H | D | CD₃ | CD₃ |
| 479 | F | H | H | H | CD₃ | CD₃ |
| 480 | F | D | D | D | CD₃ | CD₃ |
| 481 | F | D | D | D | CH₃ | CH₃ |
| 482 | F | D | D | H | CH₃ | CH₃ |
| 483 | F | D | D | H | CD₃ | CD₃ |

Specific examples of a compound of Formula I include one of the compounds of Table 19 or a pharmaceutically acceptable salt thereof, wherein V is CH; each of X^{2a}, X^{2b}, X³ and X⁴ is hydrogen, and Z is NHC(O)CD₂NHC(CD₃)₃

TABLE 19

| Cmpd | W | X^{1a} | X^{1b} | X⁵ | Y¹ | Y² |
|---|---|---|---|---|---|---|
| 484 | F | H | H | H | CH₃ | CH₃ |
| 485 | F | H | H | D | CH₃ | CH₃ |
| 486 | F | H | H | D | CD₃ | CD₃ |
| 487 | F | H | H | H | CD₃ | CD₃ |
| 488 | F | D | D | D | CD₃ | CD₃ |
| 489 | F | D | D | D | CH₃ | CH₃ |
| 490 | F | D | D | H | CH₃ | CH₃ |
| 491 | F | D | D | H | CD₃ | CD₃ |

Specific examples of a compound of Formula I include one of the compounds of Table 20 or a pharmaceutically acceptable salt thereof, wherein V is N; each of X^{2a}, X^{2b}, X³ and X⁴ is hydrogen, and Z is NH₂

TABLE 20

| Cmpd | W | X^{1a} | X^{1b} | X⁵ | Y¹ | Y² |
|---|---|---|---|---|---|---|
| 492 | F | H | H | D | CH₃ | CH₃ |
| 493 | F | H | H | D | CD₃ | CD₃ |
| 494 | F | H | H | H | CD₃ | CD₃ |
| 495 | F | D | D | D | CD₃ | CD₃ |
| 496 | F | D | D | D | CH₃ | CH₃ |
| 497 | F | D | D | H | CH₃ | CH₃ |
| 498 | F | D | D | H | CD₃ | CD₃ |
| 499 | Cl | H | H | D | CH₃ | CH₃ |
| 500 | Cl | H | H | D | CD₃ | CD₃ |
| 501 | Cl | H | H | H | CD₃ | CD₃ |
| 502 | Cl | D | D | D | CD₃ | CD₃ |
| 503 | Cl | D | D | D | CH₃ | CH₃ |
| 504 | Cl | D | D | H | CH₃ | CH₃ |
| 505 | Cl | D | D | H | CD₃ | CD₃ |
| 506 | N(CH₃)₂ | H | H | D | CH₃ | CH₃ |
| 507 | N(CH₃)₂ | H | H | D | CD₃ | CD₃ |
| 508 | N(CH₃)₂ | H | H | H | CD₃ | CD₃ |
| 509 | N(CH₃)₂ | D | D | D | CD₃ | CD₃ |
| 510 | N(CH₃)₂ | D | D | D | CH₃ | CH₃ |
| 511 | N(CH₃)₂ | D | D | H | CH₃ | CH₃ |
| 512 | N(CH₃)₂ | D | D | H | CD₃ | CD₃ |
| 513 | N(CH₃)₂ | D | D | H | CD₃ | CD₃ |
| 514 | N(CD₃)₂ | H | H | D | CH₃ | CH₃ |
| 515 | N(CD₃)₂ | H | H | D | CD₃ | CD₃ |
| 516 | N(CD₃)₂ | H | H | H | CD₃ | CD₃ |
| 517 | N(CD₃)₂ | D | D | D | CD₃ | CD₃ |
| 518 | N(CD₃)₂ | D | D | D | CH₃ | CH₃ |
| 519 | N(CD₃)₂ | D | D | H | CH₃ | CH₃ |
| 520 | N(CD₃)₂ | D | D | H | CD₃ | CD₃ |
| 521 | N(CD₃)₂ | H | H | H | CH₃ | CH₃ |

Specific examples of a compound of Formula I include one of the compounds of Table 21 or a pharmaceutically acceptable salt thereof, wherein V is N; each of X^{2a}, X^{2b}, X³ and X⁴ is hydrogen, and Z is

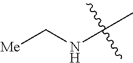 [SMILES: *NCC]

TABLE 21

| Cmpd | W | X^{1a} | X^{1b} | X⁵ | Y¹ | Y² |
|---|---|---|---|---|---|---|
| 522 | F | | | D | CH₃ | CH₃ |
| 523 | F | | | D | CD₃ | CD₃ |
| 524 | F | | | | CD₃ | CD₃ |
| 525 | F | D | D | D | CD₃ | CD₃ |
| 526 | F | D | D | D | CH₃ | CH₃ |
| 527 | F | D | D | | CH₃ | CH₃ |
| 528 | F | D | D | | CD₃ | CD₃ |
| 529 | Cl | H | H | D | CH₃ | CH₃ |
| 530 | Cl | H | H | D | CD₃ | CD₃ |
| 531 | Cl | H | H | H | CD₃ | CD₃ |
| 532 | Cl | D | D | D | CD₃ | CD₃ |
| 533 | Cl | D | D | D | CH₃ | CH₃ |
| 534 | Cl | D | D | H | CH₃ | CH₃ |
| 535 | Cl | D | D | H | CD₃ | CD₃ |
| 536 | N(CH₃)₂ | H | H | D | CH₃ | CH₃ |
| 537 | N(CH₃)₂ | H | H | D | CD₃ | CD₃ |
| 538 | N(CH₃)₂ | H | H | H | CD₃ | CD₃ |
| 539 | N(CH₃)₂ | D | D | D | CD₃ | CD₃ |
| 540 | N(CH₃)₂ | D | D | D | CH₃ | CH₃ |
| 541 | N(CH₃)₂ | D | D | H | CH₃ | CH₃ |
| 542 | N(CH₃)₂ | D | D | H | CD₃ | CD₃ |
| 543 | N(CH₃)₂ | D | D | H | CD₃ | CD₃ |
| 544 | N(CD₃)₂ | H | H | D | CH₃ | CH₃ |
| 545 | N(CD₃)₂ | H | H | D | CD₃ | CD₃ |
| 546 | N(CD₃)₂ | H | H | H | CD₃ | CD₃ |
| 547 | N(CD₃)₂ | D | D | D | CD₃ | CD₃ |
| 548 | N(CD₃)₂ | D | D | D | CH₃ | CH₃ |
| 549 | N(CD₃)₂ | D | D | H | CH₃ | CH₃ |
| 550 | N(CD₃)₂ | D | D | H | CD₃ | CD₃ |
| 551 | N(CD₃)₂ | H | H | H | CH₃ | CH₃ |

Specific examples of a compound of Formula I include one of the compounds of Table 22 or a pharmaceutically acceptable salt thereof, wherein V is N; each of X^{2a}, X^{2b}, X³ and X⁴ is hydrogen, and Z is

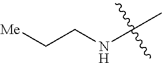 [SMILES: *NCCC]

TABLE 22

| Cmpd | W | X^{1a} | X^{1b} | X⁵ | Y¹ | Y² |
|---|---|---|---|---|---|---|
| 552 | F | H | H | D | CH₃ | CH₃ |
| 553 | F | H | H | D | CD₃ | CD₃ |
| 554 | F | H | H | H | CD₃ | CD₃ |
| 555 | F | D | D | D | CD₃ | CD₃ |
| 556 | F | D | D | D | CH₃ | CH₃ |
| 557 | F | D | D | H | CH₃ | CH₃ |
| 558 | F | D | D | H | CD₃ | CD₃ |
| 559 | Cl | H | H | D | CH₃ | CH₃ |
| 560 | Cl | H | H | D | CD₃ | CD₃ |
| 561 | Cl | H | H | H | CD₃ | CD₃ |
| 562 | Cl | D | D | D | CD₃ | CD₃ |
| 563 | Cl | D | D | D | CH₃ | CH₃ |
| 564 | Cl | D | D | H | CH₃ | CH₃ |
| 565 | Cl | D | D | H | CD₃ | CD₃ |
| 566 | N(CH₃)₂ | H | H | D | CH₃ | CH₃ |
| 567 | N(CH₃)₂ | H | H | D | CD₃ | CD₃ |
| 568 | N(CH₃)₂ | H | H | H | CD₃ | CD₃ |
| 569 | N(CH₃)₂ | D | D | D | CD₃ | CD₃ |
| 570 | N(CH₃)₂ | D | D | D | CH₃ | CH₃ |
| 571 | N(CH₃)₂ | D | D | H | CH₃ | CH₃ |
| 572 | N(CH₃)₂ | D | D | H | CD₃ | CD₃ |
| 573 | N(CH₃)₂ | D | D | H | CD₃ | CD₃ |
| 574 | N(CD₃)₂ | H | H | D | CH₃ | CH₃ |
| 575 | N(CD₃)₂ | H | H | D | CD₃ | CD₃ |
| 576 | N(CD₃)₂ | H | H | H | CD₃ | CD₃ |
| 577 | N(CD₃)₂ | D | D | D | CD₃ | CD₃ |
| 578 | N(CD₃)₂ | D | D | D | CH₃ | CH₃ |
| 579 | N(CD₃)₂ | D | D | H | CH₃ | CH₃ |
| 580 | N(CD₃)₂ | D | D | H | CD₃ | CD₃ |
| 581 | N(CD₃)₂ | H | H | H | CH₃ | CH₃ |

examples of a compound of Formula I include one of the compounds of Table 23 or a pharmaceutically acceptable salt thereof, wherein V is N; each of X^{2a}, X^{2b}, X³ and X⁴ is hydrogen, and Z is

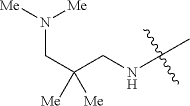 [SMILES: *NCC(C)(C)CN(C)C]

TABLE 23

| Cmpd | W | X^{1a} | X^{1b} | X⁵ | Y¹ | Y² |
|---|---|---|---|---|---|---|
| 582 | F | H | H | D | CH₃ | CH₃ |
| 583 | F | H | H | D | CD₃ | CD₃ |
| 584 | F | H | H | H | CD₃ | CD₃ |
| 585 | F | D | D | D | CD₃ | CD₃ |
| 586 | F | D | D | D | CH₃ | CH₃ |
| 587 | F | D | D | H | CH₃ | CH₃ |
| 588 | F | D | D | H | CD₃ | CD₃ |
| 589 | Cl | H | H | D | CH₃ | CH₃ |
| 590 | Cl | H | H | D | CD₃ | CD₃ |
| 591 | Cl | H | H | H | CD₃ | CD₃ |
| 592 | Cl | D | D | D | CD₃ | CD₃ |
| 593 | Cl | D | D | D | CH₃ | CH₃ |
| 594 | Cl | D | D | H | CH₃ | CH₃ |
| 595 | Cl | D | D | H | CD₃ | CD₃ |
| 596 | N(CH₃)₂ | H | H | D | CH₃ | CH₃ |
| 597 | N(CH₃)₂ | H | H | D | CD₃ | CD₃ |
| 598 | N(CH₃)₂ | H | H | H | CD₃ | CD₃ |
| 599 | N(CH₃)₂ | D | D | D | CD₃ | CD₃ |
| 600 | N(CH₃)₂ | D | D | D | CH₃ | CH₃ |
| 601 | N(CH₃)₂ | D | D | H | CH₃ | CH₃ |
| 602 | N(CH₃)₂ | D | D | H | CD₃ | CD₃ |
| 603 | N(CH₃)₂ | D | D | H | CD₃ | CD₃ |
| 604 | N(CD₃)₂ | H | H | D | CH₃ | CH₃ |
| 605 | N(CD₃)₂ | H | H | D | CD₃ | CD₃ |
| 606 | N(CD₃)₂ | H | H | H | CD₃ | CD₃ |
| 607 | N(CD₃)₂ | D | D | D | CD₃ | CD₃ |
| 608 | N(CD₃)₂ | D | D | D | CH₃ | CH₃ |
| 609 | N(CD₃)₂ | D | D | H | CH₃ | CH₃ |
| 610 | N(CD₃)₂ | D | D | H | CD₃ | CD₃ |
| 612 | N(CD₃)₂ | H | H | H | CH₃ | CH₃ |

Specific examples of a compound of Formula I include one of the compounds of Table 24 or a pharmaceutically acceptable salt thereof, wherein V is N; each of X^{2a}, X^{2b}, X³ and X⁴ is hydrogen, and Z is

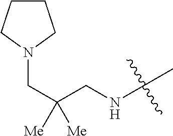 [SMILES: *NCC(C)(C)CN1CCCC1]

TABLE 24

| Cmpd | W | X^{1a} | X^{1b} | X⁵ | Y¹ | Y² |
|---|---|---|---|---|---|---|
| 613 | F | H | H | D | CH₃ | CH₃ |
| 614 | F | H | H | D | CD₃ | CD₃ |
| 615 | F | H | H | H | CD₃ | CD₃ |
| 616 | F | D | D | D | CD₃ | CD₃ |
| 617 | F | D | D | D | CH₃ | CH₃ |
| 618 | F | D | D | H | CH₃ | CH₃ |
| 619 | F | D | D | H | CD₃ | CD₃ |
| 620 | Cl | H | H | D | CH₃ | CH₃ |
| 621 | Cl | H | H | D | CD₃ | CD₃ |
| 622 | Cl | H | H | H | CD₃ | CD₃ |
| 623 | Cl | D | D | D | CD₃ | CD₃ |
| 624 | Cl | D | D | D | CH₃ | CH₃ |
| 625 | Cl | D | D | H | CH₃ | CH₃ |
| 626 | Cl | D | D | H | CD₃ | CD₃ |
| 627 | N(CH₃)₂ | H | H | D | CH₃ | CH₃ |
| 628 | N(CH₃)₂ | H | H | D | CD₃ | CD₃ |
| 629 | N(CH₃)₂ | H | H | H | CD₃ | CD₃ |
| 630 | N(CH₃)₂ | D | D | D | CD₃ | CD₃ |
| 631 | N(CH₃)₂ | D | D | D | CH₃ | CH₃ |
| 632 | N(CH₃)₂ | D | D | H | CH₃ | CH₃ |
| 633 | N(CH₃)₂ | D | D | H | CD₃ | CD₃ |
| 634 | N(CH₃)₂ | D | D | H | CD₃ | CD₃ |
| 635 | N(CD₃)₂ | H | H | D | CH₃ | CH₃ |
| 636 | N(CD₃)₂ | H | H | D | CD₃ | CD₃ |
| 637 | N(CD₃)₂ | H | H | H | CD₃ | CD₃ |
| 638 | N(CD₃)₂ | D | D | D | CD₃ | CD₃ |
| 639 | N(CD₃)₂ | D | D | D | CH₃ | CH₃ |
| 640 | N(CD₃)₂ | D | D | H | CH₃ | CH₃ |
| 641 | N(CD₃)₂ | D | D | H | CD₃ | CD₃ |
| 642 | N(CD₃)₂ | H | H | H | CH₃ | CH₃ |

The synthesis of compounds of Formula I and Ia may be readily achieved by synthetic chemists of ordinary skill by reference to the Exemplary Synthesis disclosed herein. The synthesis of compounds of Formula I can be readily achieved by synthetic chemists of ordinary skill. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Procedures and intermediates relevant to the preparation of the corresponding non-deuterated compound are disclosed, for instance in PCT publication WO 2010/017470 A1, Charest, M. G., et al., Science 2005, 308, pp. 395-398, Sun, C., et al., J. Am. Chem. Soc. 2008, pp. 17913-17927, Xiao, X., et al., 49^{th} Annual ICAAC, Sep. 12-15, 2009, F1-1514, Lofland, D., et al., 49^{th} Annual ICAAC, Sep. 12-15, 2009, F1-1515, and in PCT publication WO 2009/009042 A1.

### EXEMPLARY SYNTHESIS

A convenient method for synthesizing compounds of Formula I is depicted in Scheme 1:

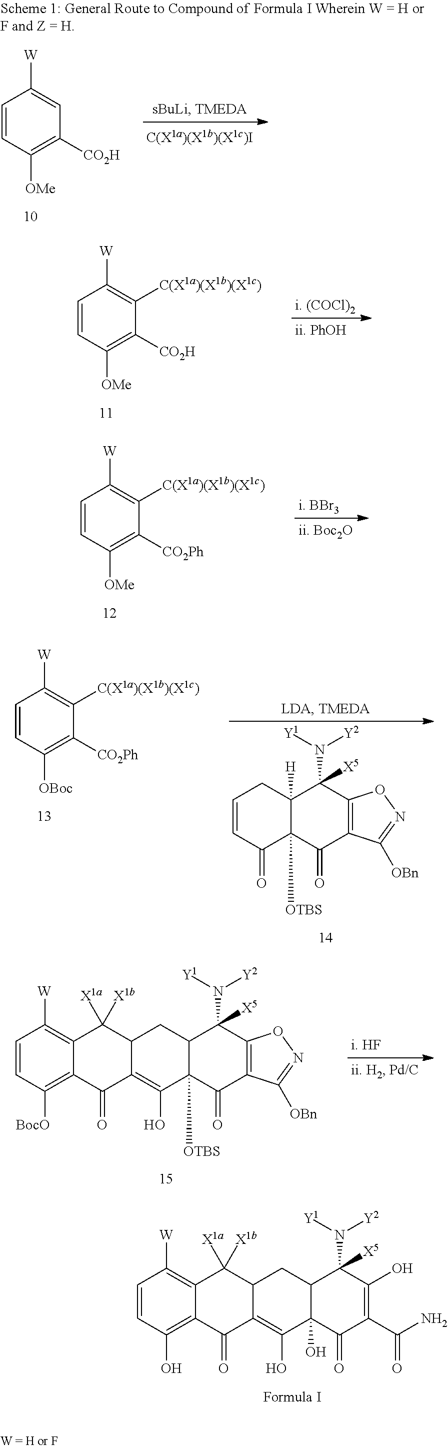

Scheme 1 depicts a route to compounds of Formula I where V═CH, W═F or H, and Z═H. These may be assembled employing intermediate 14 in a manner analogous to the one described in WO 2010/017470. Aryl methylation of 10 (W═H or F) with either iodomethane or d3-iodomethane followed by acid chloride formation and subsequent treatment with phenol affords the appropriately deuterated benzoic acid phenyl ester 12. The tert-butyl carbonate 13 is then obtained via demethylation and subsequent Boc protection. The benzylic anion formed upon treatment with LDA is then exposed to intermediate 14, effecting cyclization to the isoxazole-capped tetracycline core 15. Simultaneous removal of the silyl and Boc protecting groups with HF is followed by reductive deprotection of the benzyl ether providing deuterated analogs of Formula I where V═CH, W═F or H, and Z═H.

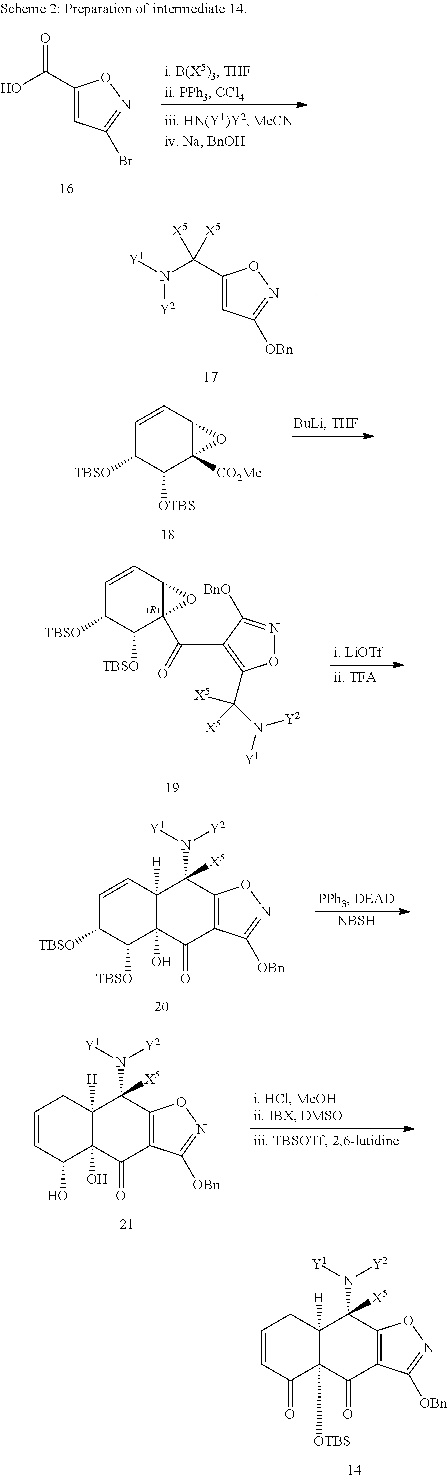

Scheme 2 depicts a route to intermediate 14. Treatment of 3-bromoisoxazole-5-carboxylic acid 16 with borane-THF or d3-borane-THF in a manner analogous to the procedure described in WO 2008/0103130 affords the appropriately deuterated alcohol which is then converted to the corresponding chloride in a manner analogous to the one of WO 2008/042571. Displacement of the chloride with either dimethylamine or d6-dimethylamine followed by displacement of the C-3 bromide with benzyl alcohol affords the appropriately deuterated dimethylamino benzyl ether 17 analogously to what is described in WO 2007/117639 A2. The remaining steps for the completion of the synthesis of 14, including preparation of the epoxy methyl ester 18, employ procedures analogous to the ones described in Charest, M. G., et al., Science 2005, pp. 395-398: Isoxazole deprotonation followed by treatment with 18 provides the epoxy ketone 19 which is subsequently cyclized upon exposure to lithium triflate. Migration of the double bond followed by silyl deprotection, IBX mediated alcohol oxidation, and subsequent TBS protection of the tertiary alcohol moiety ultimately affords appropriately deuterated 14.

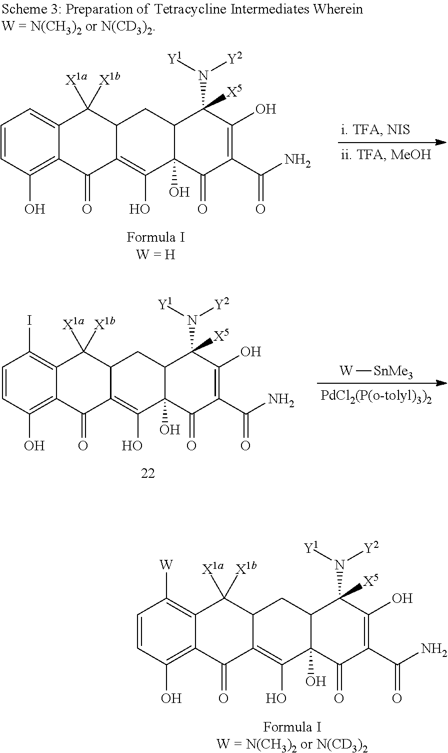

Scheme 3 depicts a route to compounds of Formula I where V═CH, W═N(CH₃)₂ or N(CD₃)₂ and Z═H. These may be assembled starting from compounds of Formula I wherein W═H. Analogously to Nelson, M. L. et al., J. Org. Chem. 2003, pp. 5838-5851, regioselective iodination to afford 22 may be achieved upon treatment of compounds of Formula I wherein W═H with N-iodosuccinimide in the presence of trifluoroacetic acid. Installation of either dimethylamine or d6-dimethylamine is then achieved via palladium catalyzed cross-coupling of commercially available Me₃Sn—N(CH₃)₂ or in-situ generated Me₃Sn—N(CD₃)₂ in a manner analogous to Koza, D. J. et al., J. Org. Chem. 2002, pp. 5025-5027. Preparation of the aminostannane follows protocols described in: Buchwald, S. L. et al. J. Am. Chem. Soc. 1994, pp. 7901-7902 while facile preparation of the required palladium catalyst is described in Tanner, D. et al., J. Org. Chem, 2002, pp. 6367-6371.

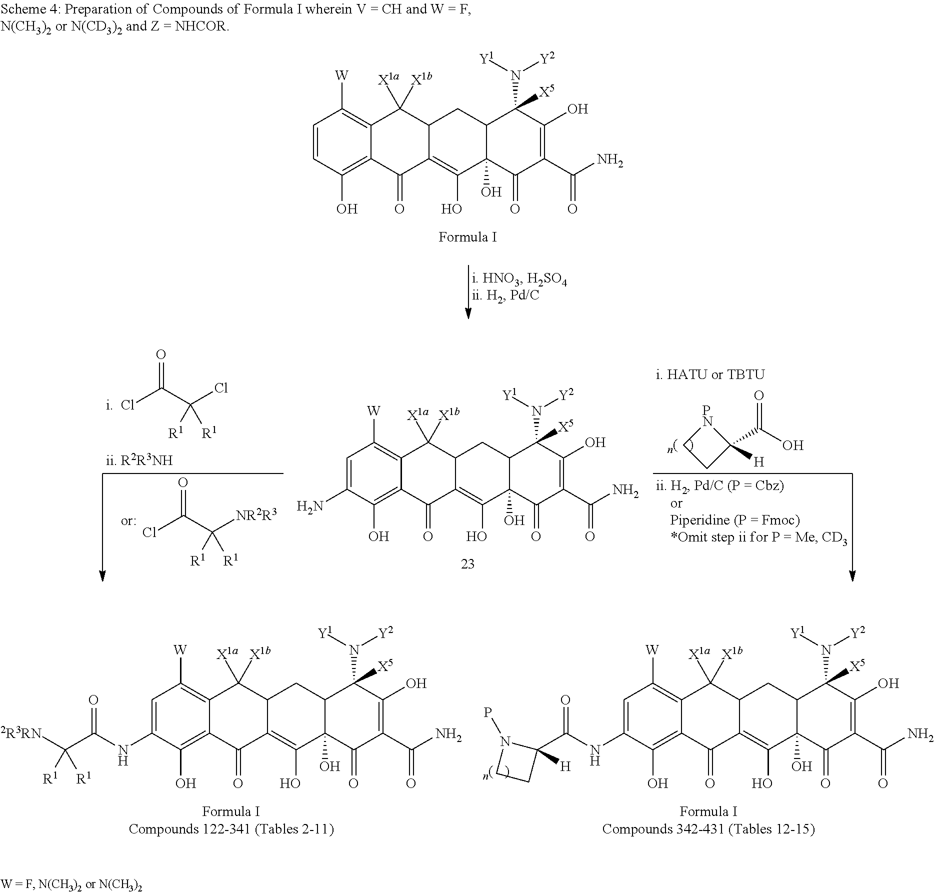

Scheme 4 depicts a route to compounds of Formula I where V═CH and W═F, N(CH₃)₂ or N(CD₃)₂ and Z═NHCOR. These may be accessed starting from the appropriate compounds of Formula I wherein W═F, N(CH₃)₂ or N(CD₃)₂ analogously to the procedures described in WO 2010/017470 A1. Regioselective nitration of the aryl ring followed by palladium catalyzed hydrogenation affords the aryl amine 23. Compounds of Formula I are then obtained by treatment of 23 with either chloroacetyl chloride or d2-chloroacetyl chloride followed by displacement with an appropriately labeled amine or by direct acetylation with an appropriate acid chloride. Alternatively, cyclic aminoacids may be coupled to the aryl amine using common coupling reagents. In Scheme 4, such a cyclic amino acid is depicted where P is (a) CH₃ or CD₃, or (b) a protecting group such as Cbz or Fmoc. In case (b), Cbz or Fmoc protecting groups are subsequently removed in order to access the final compounds.

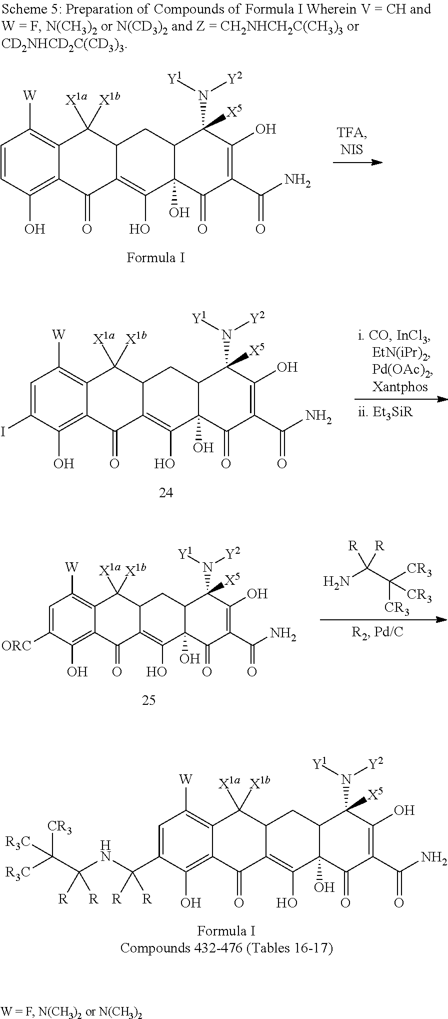

Scheme 5 depicts a route to compounds of Formula I where V═CH, W═F, N(CH₃)₂ or N(CD₃)₂ and Z═CH₂NHCH₂C(CH₃)₃ or CD₂NHCD₂C(CD₃)₃. Iodination of the appropriate compound of Formula I (where W═F, N(CH₃)₂ or N(CD₃)₂) to afford 24 is achieved with N-iodosuccinimide following the procedure analogous to the one described in Nelson, M. L. et al., J. Org. Chem. 2003, pp. 5838-5851. Employing protocols analogous to the ones of WO 2009/009042, the aryl iodide is then formylated employing either triethylsilane or d1-triethylsilane then subsequently reacted with either neopentylamine or d11-neopentylamine under reductive amination conditions to afford the desired compounds of Formula I.

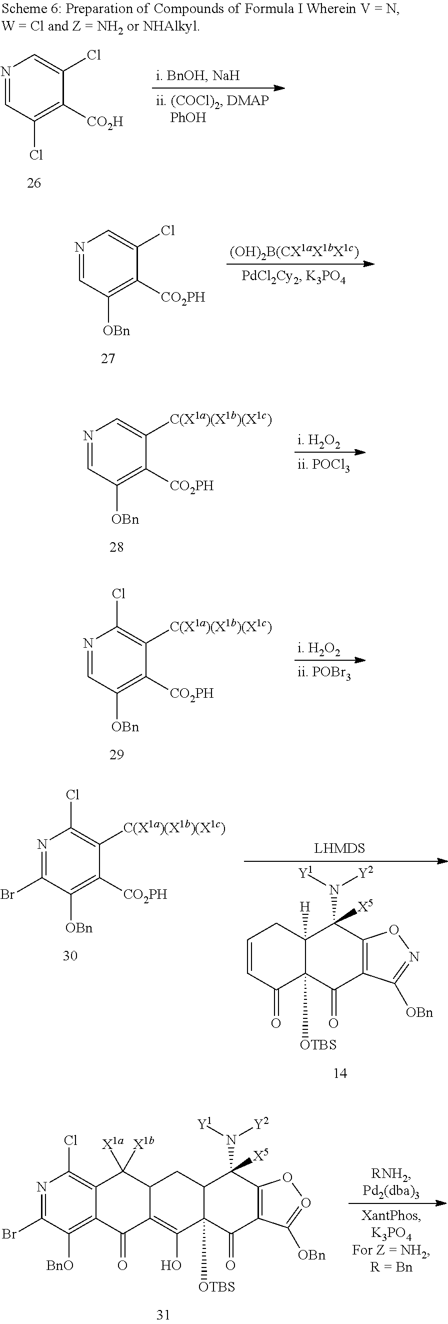

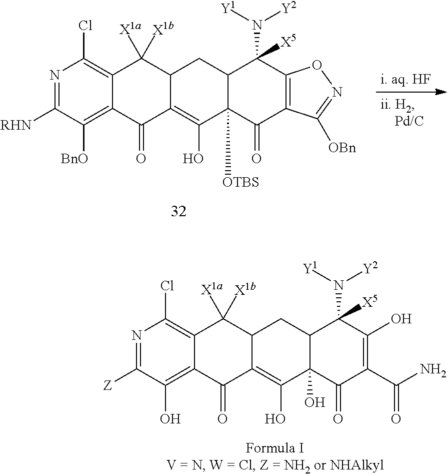

Scheme 6 depicts a route to compounds of Formula I where V═N, W═Cl and Z═NH₂ or NHAlkyl, which may be obtained analogously to the procedure described in the following poster: F1-1514, 49^{th} Annual ICAAC, Sep. 12-15, 2009, San Francisco, Calif. Reaction of 26 with benzyl alcohol in the presence of sodium hydride affords 3-chloro-5-benzyloxy-isonicotinic acid which is then converted to the phenyl ester 27. Cross-coupling with either CH₃B(OH)₂ or CD₃B(OH)₂ followed by 2-step chlorination with hydrogen peroxide and phosphorous oxychloride affords the suitably deuterated 2-chloro-isonicotinic acid phenyl ester analog 29. The bromide is then installed at the 6-position employing a similar 2-step protocol. The benzylic anion formed upon treatment with LHMDS is then exposed to 14, effecting cyclization to the isoxazole-capped azatetracycline core. Palladium catalyzed amination affords 32. Silyl deprotection and subsequent hydrogenolysis of the benzyl moiety ultimately furnishes the desired compounds of Formula I where V═N, W═Cl, and Z═NH₂ or NHAlkyl. When Z═NH₂ in the scheme, benzylamine is the amine RNH₂ that is coupled during the amination step, the benzyl group being then cleaved to afford the free amine during the final hydrogenolysis.

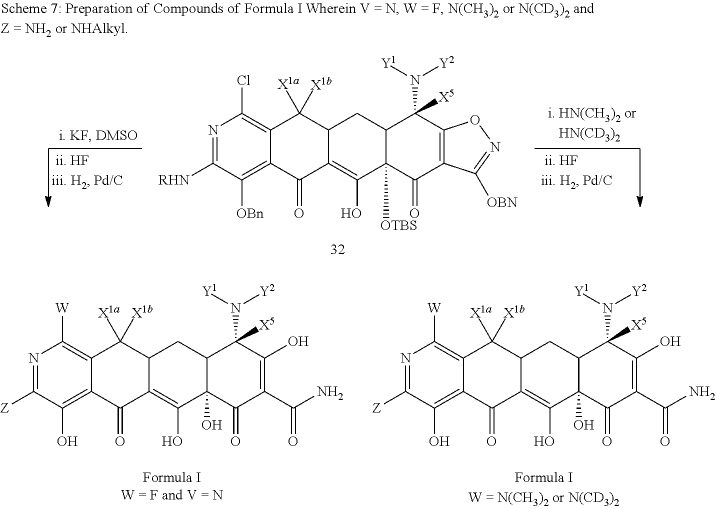

Scheme 7 depicts the conversion of 32 (prepared as described in Scheme 6 above) into compounds of Formula I where V═N, W═F, N(CH₃)₂ or N(CD₃)₂ and Z═NH₂ or NHAlkyl. Reaction of 32 with potassium fluoride in DMSO at an elevated temperature (analogously to Shestopalov, A. M. et al., J. Fluorine Chem., 2009, pp. 236-240) results in displacement of the chloride atom with fluoride. Hydrogen fluoride-mediated silyl deprotection followed by hydrogenolysis furnishes the desired compounds of Formula I where V═N, W═F, and Z═NH₂ or NHAlkyl. In a similar manner, treatment of 32 with either dimethylamine or d6-dimethylamine in the presence of a trialkylamine base (analogously to Cox, J. M. et al., Bioorg. Med. Chem. Lett., 2007, pp. 4579-4583) results in displacement of the chloride atom with (d6)-dimethylamine. Silyl deprotection and hydrogenolysis furnish the desired compounds of Formula I where V═N, W═N(CH₃)₂ or N(CD₃)₂, and Z═NH₂ or NHAlkyl. As in Scheme 6 above, Z═NH₂ is obtained in the case where R=benzyl, the benzyl group being then cleaved to afford the free amine during the final hydrogenolysis.

Unless otherwise stated, all reactants and reagents discussed in Schemes 1-7 above are commercially available with the exception of the following: The preparation of (S)—N-methyl-2-azetidine carboxylic acid 33 and (S)—N-d3-methyl-2-azetidine 34 follows the procedures described in: Takhi, M. et al., WO 2009/032326 A1 and involves palladium catalyzed reductive amination of (S)-2-azetidine carboxylic acid with formaldehyde under an atmosphere of H₂ or d2-formaldehyde under an atmosphere of D₂. Synthesis of d11-neopentylamine 35 involves conversion of d9-pivalic acid into d9-pivalamide (following Kaufmann, D. et al., J. Med. Chem., 2009, pp. 7236-7248) followed by treatment with lithium aluminum deuteride to facilitate reduction to the primary amine.

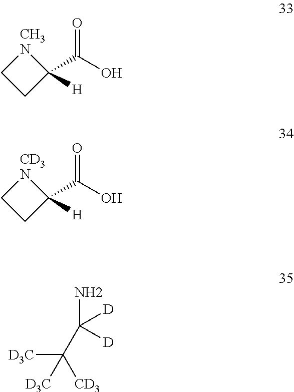

The following commercially available compounds may be used as synthetic reagents or intermediates in Schemes 1-7 above, and/or to prepare the synthetic reagents or intermediates that may be used in Schemes 1-7.

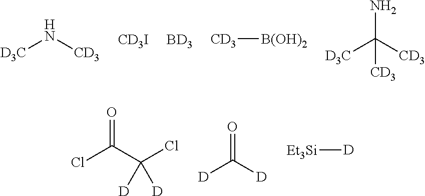

For embodiments such as those disclosed in Tables 8b-8g, suitably deuterated pyrrolidines are either commercially available or may be prepared from commercially available pyrrolidines. In particular, the two deuterated pyrrolidines shown below are commercially available:

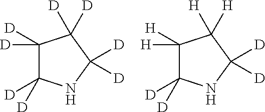

The pyrrolidine shown below

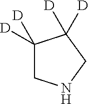

may be obtained according to the following scheme:

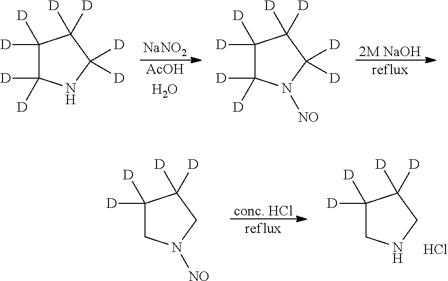

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Combinations of substituents and variables envisioned by this invention are only those that result in the formation of stable compounds.

### Compositions

The invention also provides pyrogen-free compositions comprising a compound of Formula I or Ia, or a pharmaceutically acceptable salt of said compound; and an acceptable carrier. In one embodiment, the composition comprises an effective amount of the compound of Formula I or Ia. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

In another embodiment, a composition of this invention further comprises a second therapeutic agent. Preferably, the second therapeutic agent is an agent useful in the treatment or prevention of a disease or condition that may be treated by the administration of a tetracycline, such as a compound of the invention.

In an exemplary embodiment, the condition is selected from cIAI (Complicated Intra-abdominal Infections), HAP (Hospital Associated Pneumonia), VAP (Ventilator Associated Pneumonia), cSSSI (complicated skin and skin-structure infections), cUTI (complicated urinary tract infections) and CABP (community acquired bacterial pneumonia).

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In one embodiment of the pharmaceutical compositions of the invention, the compound of the present invention is present in an effective amount. As used herein, the term “effective amount” refers to an amount which, when administered in a proper dosing regimen, is sufficient to treat a disease or disorder, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.01 to about 5000 mg per treatment. In more specific embodiments the range is from about 0.1 to 2500 mg, or from 0.2 to 1000 mg, or most specifically from about 1 to 500 mg. Treatment typically is administered one to three times daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

### Methods of Treatment

According to another embodiment, the invention provides a method of treating a patient suffering from, or susceptible to, a disease or condition that is beneficially treated by a tetracycline comprising the step of administering to said patient an effective amount of a compound of this invention or a pharmaceutically acceptable salt of said compound or a composition of this invention.

Such conditions include bacterial, viral, parasitic, and fungal infections (including those which are resistant to other tetracycline compounds), cancer (e.g., prostate, breast, colon, lung melanoma and lymph cancers and other disorders characterized by unwanted cellular proliferation), arthritis, osteoporosis, diabetes, stroke, AMI, aortic aneurysm, and neurodegenerative diseases. Such conditions alos include diarrhea, urinary tract infections, infections of skin and skin structure, ear, nose and throat infections, wound infection, mastitis and the like. In addition, methods for treating neoplasms using tetracycline compounds of the invention are also included (van der Bozert et al., Cancer Res., 48:6686-6690 (1988)). In one embodiment, the condition is not a bacterial infection.

Such conditions also include conditions in which inflammation or inflammatory factors (such as matrix metalloproteinases (MMPs), nitric oxide (NO), TNF, interleukins, plasma proteins, cellular defense systems, cytokines, lipid metabolites, proteases, toxic radicals, and/or adhesion molecules), are involved or are present in an area in aberrant amounts. Such conditions also include conditions in which there is an increase in acute phase proteins (e.g., C-reactive protein). The cause of inflammation may be due to physical damage, chemical substances, micro-organisms, tissue necrosis, cancer or other agents.

Such conditions also include inflammatory disorders, such as inflammatory disorders caused by microbial infections (e.g., bacterial and fungal infections), physical agents (e.g., burns, radiation, and trauma), chemical agents (e.g., toxins and caustic substances), tissue necrosis and various types of immunologic reactions. Examples of inflammatory disorders include osteoarthritis, rheumatoid arthritis, acute and chronic infections (bacterial and fungal, including diphtheria and pertussis); acute and chronic bronchitis, sinusitis, and upper respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; animal bites (e.g., spider bites, snake bites, insect bites and the like); burns (thermal, chemical, and electrical); inflammatory bowel disorder (DBD); common obstructive pulmonary disease (COPD); acute respiratory distress syndrome (ARDS); vasculitis; asthma; sepsis; nephritis; pancreatitis; hepatitis; lupus; viral infections; parasitic infections; and sunburn.

Such conditions also include conditions which involve or are associated with nitric oxide (NO) or inducible nitric oxide synthase (iNOS). NO associated state includes states which are characterized by aberrant amounts of NO and/or iNOS. Preferably, the NO associated state can be treated by administering tetracycline compounds of the invention.

Such conditions also include conditions malaria, senescence, diabetes, vascular stroke, hemorrhagic stroke, neurodegenerative disorders (Alzheimer's disease & Huntingdon's disease), cardiac disease (reperfusion-associated injury following infarction), juvenile diabetes, inflammatory disorders, osteoarthritis, rheumatoid arthritis, acute, recurrent and chronic infections (bacterial, viral and fungal); acute and chronic bronchitis, sinusitis, and respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendonitis); uremic pericarditis; acute and chronic cholecystis; cystic fibrosis, acute and chronic vaginitis; acute and chronic uveitis; drug reactions; insect bites; burns (thermal, chemical, and electrical); and sunburn.

Such conditions also include conditions arteriosclerosis, angiogenesis, corneal ulceration, emphysema, osteoarthritis, multiple sclerosis (Liedtke et al, Ann. Neurol. 1998, 44:35-46; Chandler et al, J. Neuroimmunol. 1997, 72:155-71), osteosarcoma, osteomyelitis, bronchiectasis, chronic pulmonary obstructive disease, skin and eye diseases, periodontitis, osteoporosis, rheumatoid arthritis, ulcerative colitis, inflammatory disorders, tumor growth and invasion (Stetler-Stevenson et al, Annu. Rev. Cell Biol. 1993, 9:541-73; Tryggvason et al, Biochim. Biophys. Acta 1987, 907: 191-217; Li et al, MoI. Carcinog. 1998, 22:84-89)), metastasis, acute lung injury, stroke, ischemia, diabetes, aortic or vascular aneurysms, skin tissue wounds, dry eye, bone and cartilage degradation (Greenwald et al, Bone 1998, 22:33-38; Ryan et al, Curr. Op. Rheumatol. 1996, 8; 238-247).

Such conditions also include cancer such as all solid tumors, i.e., carcinomas e.g., adenocarcinomas, and sarcomas, as well as cancer growth in adenocarcinomas. Examples of carcinomas include carcinomas of the prostate, breast, ovary, testis, lung, colon, and breast. Examples of cancers also include any solid tumor derived from any organ system. Examples of cancers also include colon cancer, bladder cancer, breast cancer, melanoma, ovarian carcinoma, prostatic carcinoma, lung cancer, and a variety of other cancers as well.

Such conditions also include neurological disorders which include both neuropsychiatric and neurodegenerative disorders, such as Alzheimer's disease, dementias related to Alzheimer's disease (such as Pick's disease), Parkinson's and other Lewy diffuse body diseases, senile dementia, Huntington's disease, Gilles de Ia Tourette's syndrome, multiple sclerosis (e.g., including but not limited to, relapsing and remitting multiple sclerosis, primary progressive multiple sclerosis, and secondary progressive multiple sclerosis), amyotrophic lateral sclerosis (ALS), progressive supranuclear palsy, epilepsy, and Creutzfeldt-Jakob disease; autonomic function disorders such as hypertension and sleep disorders, and neuropsychiatric disorders, such as depression, schizophrenia, schizoaffective disorder, Korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-I), bipolar affective neurological disorders, e.g., migraine and obesity.

Such conditions also include diabetes, e.g., juvenile diabetes, diabetes mellitus, diabetes type I, or diabetes type II. In a further embodiment, protein glycosylation is not affected by the administration of the compounds of the invention. In another embodiment, the compound of the invention is administered in combination with standard diabetic therapies, such as, but not limited to insulin therapy.

Such conditions also include bone mass disorders. Such as osteoporosis (e.g., a decrease in bone strength and density), bone fractures, bone formation associated with surgical procedures {e.g., facial reconstruction), osteogenesis imperfecta (brittle bone disease), hypophosphatasia, Paget's disease, fibrous dysplasia, osteopetrosis, myeloma bone disease, and the depletion of calcium in bone, such as that which is related to primary hyperparathyroidism.

Such conditions also include acute lung injury or injuries, which include adult respiratory distress syndrome (ARDS), post-pump syndrome (PPS), adelectasis (e.g., collapsed lung) and trauma. Trauma includes any injury to living tissue caused by an extrinsic agent or event. Examples of trauma include crush injuries, contact with a hard surface, or cutting or other damage to the lungs. Such conditions also include chronic lung disorders, which include asthma, chronic obstructive pulmonary disease (COPD), cystic fibrosis, and emphesema.

Such conditions also include ischemia, stroke, hemorrhagic stroke or ischemic stroke.

Such conditions also include a skin wound or wounds. The invention also provides a method for improving the healing response of the epithelialized tissue (e.g., skin, mucusae) to acute traumatic injury (e.g., cut, burn, scrape, etc.).

Such conditions also include an aortic or vascular aneurysm in vascular tissue of a subject (e.g., a subject having or at risk of having an aortic or vascular aneurysm, etc.). In one embodiment, the vascular tissue is an artery, e.g., the aorta, e.g., the abdominal aorta.

Such conditions also include bacterial infections caused by a wide variety of gram positive and gram negative bacteria, including rickettsiae; a number of gram-positive and gram-negative bacteria; and the agents responsible for lymphogranuloma venereum, inclusion conjunctivitis, or psittacosis. Such conditions also include infections of, e.g., K. pneumoniae, Salmonella, K hirae, A. baumanii, B. catarrhalis, H. influenzae, P. aeruginosa, E. faecium, E. coli, S. aureus or E. faecalis.

Such conditions also include a bacterial infection that is resistant to other tetracycline antibiotic compounds.

In an exemplary embodiment, the condition is selected from cIAI (Complicated Intra-abdominal Infections), HAP (Hospital Associated Pneumonia), VAP (Ventilator Associated Pneumonia), cSSSI (complicated skin and skin-structure infections), cUTI (complicated urinary tract infections) and CABP (community acquired bacterial pneumonia).

In another embodiment, any of the above methods of treatment comprises the further step of co-administering to the patient one or more second therapeutic agents. The choice of second therapeutic agent may be made from any second therapeutic agent known to be useful for co-administration with a compound that treats any condition disclosed herein. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered (i) together with a compound of this invention as part of a single dosage form (such as a composition of this invention comprising a compound of the invention and an second therapeutic agent as described above) or as separate, multiple dosage forms; or (ii) prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I or Ia, or a pharmaceutically acceptable salt of said compound, alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I or Ia or a pharmaceutically acceptable salt thereof for use in the treatment or prevention in a patient of a disease, disorder or symptom thereof delineated herein.

### EXAMPLES

### Example 1

### Evaluation of Metabolic Stability in Human Liver Microsomes

Human liver microsomes (20 mg/mL) are available from Xenotech, LLC (Lenexa, Kans.). β-nicotinamide adenine dinucleotide phosphate, reduced form (NADPH), magnesium chloride (MgCl₂), and dimethyl sulfoxide (DMSO) are available from Sigma-Aldrich.

7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated tetracycline corresponding to the compound of the invention and the positive control, 7-ethoxycoumarin (1 μM). Testing is done in triplicate.

The in vitro t_{1/2}s for test compounds are calculated from the slopes of the linear regression of % parent remaining (ln) vs incubation time relationship:

in vitro t_{1/2}=0.693/k

k=−[slope of linear regression of % parent remaining (ln) vs incubation time]. Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 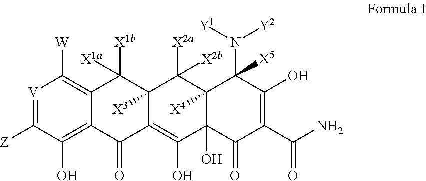
or a pharmaceutically acceptable salt thereof, wherein:
V is CH or N;
W is F;
each X is independently selected from H and D;
each Y is independently selected from CH₃ and CD₃;
Z is H; NH₂; C₁-C₆ alkylene-R¹ wherein the C₁-C₆ alkylene is optionally substituted with deuterium; or NHQR¹;
Q is —C(O)— or a direct bond;
R¹ is C₀-C₆ alkyl optionally substituted with deuterium and optionally substituted with R⁴; or NH(C₁-C₆ alkyl) wherein the C₁-C₆ alkyl is optionally substituted with deuterium;
R⁴ is NR²R³; or a 3- to 10-membered heterocyclyl containing at least one ring nitrogen wherein the 3- to 10-membered heterocyclyl is optionally substituted with deuterium at a carbon atom and is optionally substituted with C₁-C₆ alkyl that is optionally substituted with deuterium;
each of R² and R³ is independently H; C₁-C₆ alkyl; (C₃-C₈ cycloalkyl); or (C₀-C₂)alkylene(C₃-C₈ cycloalkyl); wherein each C₁-C₆ alkyl, (C₃-C₈ cycloalkyl), and (C₀-C₂)alkylene(C₃-C₈ cycloalkyl) of R² and R³ is independently optionally substituted with deuterium;
with the proviso that if W is other than CD₃, OCD₃ or N(CD₃)₂; each X is H; and each Y is CH₃;
then Z comprises deuterium;
and with the proviso that if W is N(CH₃)₂ or N(CD₃)₂, then X⁵ is D and at least one of X^{1a}, X^{1b}, X^{2a}, X^{2b}, X³ and X⁴ is hydrogen.

2. A compound of claim 1, wherein X^{1a} and X^{1b} are the same; X^{2a} and X^{2b} are the same; Y¹ and Y² are the same; and Z is H; NH₂; or C₁-C₅ alkylene-R¹ where the C₁-C₅ alkylene is optionally substituted with deuterium; or NHQR¹.

3. A compound of claim 1 or 2, wherein X⁵ is deuterium.

4. A compound of claim 1, wherein X^{1a} and X^{1b} are each hydrogen.

5. A compound of claim 1, wherein X^{1a} and X^{1b} are each deuterium.

6. A compound of claim 1, wherein V is CH.

7. A compound of claim 6, wherein W is F; X^{1a} and X^{1b} are the same; X^{2a} and X^{2b} are the same; and Y¹ and Y² are the same.

8. A compound of claim 7, wherein X^{1a}, X^{1b}, X^{2a} and X^{2b} are each hydrogen.

9. A compound of claim 8, wherein Z is H, CH₂NHCH₂C(CH₃)₃, NHCOCH₂C(CH₃)₃, NHCOCH₂NHCH₂CH₃, NHCOCH₂NHCH₂CH₂CH₃, NHCOCH₂NHCH₂CH₂CH₂CH₃, NHCOCH₂NH-cyclopentyl, NHCOCH₂NH-cyclobutyl, NHCOCH₂NHCH₂-cyclopropyl, NHCOCH₂NHCH₂-cyclobutyl, NHC(O)CH₂N(CH₃)₂, NHC(O)CH₂N(CH₂CH₃)(CH₃), NHC(O)CH₂(1-pyrrolidyl), NHCO—(S)-2-pyrrolidyl, NHCO—(S)-2-azetidinyl, NHCO—(S)-2-(N-methyl)-azetidyl, or —NHCO—(S)-2-(N-methyl)-pyrrolidyl, wherein Z is optionally substituted with deuterium.

10. A compound of claim 1, wherein V is N.

11. A compound of claim 10, wherein W is F; X^{1a} and X^{1b} are the same; X^{2a} and X^{2b} are the same; and Y¹ and Y² are the same.

12. A compound of claim 11, wherein X^{1a}, X^{1b}, X^{2a} and X^{2b} are each hydrogen.

13. A compound of claim 12, wherein Z is NH₂, NHCH₂CH₃, NHCH₂CH₂CH₃, NHCH₂C(CH₃)₂CH₂N(CH₃)₂, or NHCH₂C(CH₃)₂CH₂(1-pyrrolidyl), wherein Z is optionally substituted with deuterium.

14. The compound of claim 1, wherein the compound of formula I is a compound of formula Ia 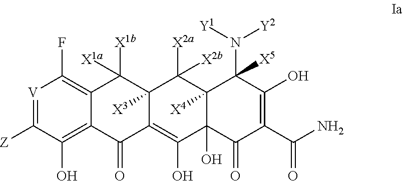
or pharmaceutically acceptable salt thereof.

15. The compound of claim 1, wherein any atom not designated as deuterium is present at its natural isotopic abundance

16. A pyrogen-free pharmaceutical composition comprising a compound of claim 1 or a pharmaceutically acceptable salt of said compound; and a pharmaceutically acceptable carrier.

17. The composition of claim 16 additionally comprising a second therapeutic agent useful in the treatment or prevention of a disease or condition selected from cIAI, HAP, VAP, cSSSI, cUTI and CABP.

18. A method of treating a patient suffering from, or susceptible to, a disease or condition selected from cIAI, HAP, VAP, cSSSI, cUTI and CABP, comprising the step of administering to the patient in need thereof an effective amount of a composition of claim 16.

19. The compound of claim 1, wherein V is CH; each of X^{2a}, X^{2b}, X³ and X⁴ is hydrogen; Z is 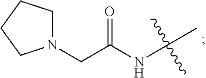 [SMILES: *NC(=O)CN1CCCC1]
and wherein the compound is selected from any of the compounds (Cmpd) set forth in the table below:

| Cmpd | W | X^{1a} | X^{1b} | X⁵ | Y¹ | Y² |
|---|---|---|---|---|---|---|
| 254a | F | H | H | D | CH₃ | CH₃ |
| 255a | F | H | H | D | CD₃ | CD₃ |
| 256a | F | H | H | H | CD₃ | CD₃ |
| 257a | F | D | D | D | CD₃ | CD₃ |
| 258a | F | D | D | D | CH₃ | CH₃ |
| 259a | F | D | D | H | CH₃ | CH₃ |
| 260a | F | D | D | H | CD₃ | CD₃ |

or a pharmaceutically acceptable salt thereof, wherein any atom not designated as deuterium is present at its natural isotopic abundance.

20. The compound of claim 1, wherein V is CH; each of X^{2a}, X^{2b}, X³ and X⁴ is hydrogen; Z is 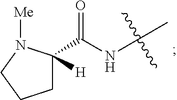 [SMILES: *NC(=O)[C@@H]1CCCN1C]
and wherein the compound is selected from any of the compounds (Cmpd) set forth in the table below:

| Cmpd | W | X^{1a} | X^{1b} | X⁵ | Y¹ | Y² |
|---|---|---|---|---|---|---|
| 408a | F | H | H | H | CH₃ | CH₃ |
| 409a | F | H | H | D | CH₃ | CH₃ |
| 410a | F | H | H | D | CD₃ | CD₃ |
| 411a | F | H | H | H | CD₃ | CD₃ |
| 412a | F | D | D | D | CD₃ | CD₃ |
| 413a | F | D | D | D | CH₃ | CH₃ |
| 414a | F | D | D | H | CH₃ | CH₃ |
| 415a | F | D | D | H | CD₃ | CD₃ |

or a pharmaceutically acceptable salt thereof, wherein any atom not designated as deuterium is present at its natural isotopic abundance.
